(19) United States
(12) Patent Application Publication
(10) Pub. No.: US 2022/0186297 A1
(43) Pub. Date: Jun. 16, 2022

(54) ULTRASENSITIVE BIOSENSOR METHODS

(71) Applicant: Quantum-Si Incorporated, Guilford, CT (US)

(72) Inventors: Brian Reed, Madison, CT (US); An Li, Guilford, CT (US); Omer Ad, Madison, CT (US); Haidong Huang, Madison, CT (US); Guojun Chen, Sherborn, MA (US)

(73) Assignee: Quantum-Si Incorporated, Guilford, CT (US)

(21) Appl. No.: 17/552,339

(22) Filed: Dec. 15, 2021

Related U.S. Application Data
(60) Provisional application No. 63/125,634, filed on Dec. 15, 2020.

(51) Int. Cl. C12Q 1/6823; C12Q 1/6837; C12Q 1/6876; G01N 33/543; G01N 33/58; C12Q 1/6804

(52) U.S. Cl. CPC C12Q 1/6823; C12Q 1/6837; C12Q 1/6876; G01N 33/54366; G01N 2496/15; G01N 33/582; C12Q 1/6804; C12Q 2600/166; G01N 33/54306

(57) ABSTRACT

Methods and devices for ultrasensitive detection of target molecules (e.g., target nucleic acids or target proteins) from a biological sample are provided herein. In some embodiments, methods and devices enable ultrasensitive determination of the concentration of target molecules.

### CROSS-REFERENCE TO RELATED APPLICATION

This application claims priority under 35 U.S.C. § 119(e) to U.S. Provisional Patent Application No. 63/125,634, filed Dec. 15, 2020, which is hereby incorporated by reference in its entirety.

### BACKGROUND

Methods and systems that are able to quickly and accurately detect and, in certain cases, quantify a target analyte molecule in a sample are essential analytical measurements for academic and industrial research, environmental assessment, food safety, medical diagnosis, and detection of chemical, biological, and/or radiological warfare agents. Most previous techniques for quantifying low levels of analyte molecules in a sample use amplification procedures to increase the number of reporter molecules in order to be able to provide a measurable signal. For example, these known methods include enzyme-linked immunosorbent assays (ELISA) for amplifying the signal in antibody-based assays, as well as the polymerase chain reaction (PCR) for amplifying target DNA strands in DNA-based assays.

These known methods and/or systems are based on ensemble responses in which many analyte molecules give rise to a measured signal. Most detection schemes require that a large number of molecules are present in the ensemble for the aggregate signal to be above the detection threshold. This requirement limits the sensitivity of most detection techniques and the dynamic range (e.g., the range of concentrations that can be detected). Many of the known methods are further plagued with problems of non-specific binding, which can lead to an increase in the background signal, and therefore limits the lowest concentration that may be accurately or reproducibly detected.

Accordingly, improved methods for detecting and quantifying target molecules in a sample are needed, especially in samples where such molecules or particles are present at very low concentration.

### SUMMARY

Aspects of the instant disclosure provide methods, compositions, devices, and/or cartridge or detection chips for use in a process, e.g., to determine the amount (e.g., concentration) of a target molecule in a sample. Some aspects of the disclosure provide a method of determining the concentration of target molecules (e.g., labeled target molecules) in a sample.

In some embodiments, a method of determining the concentration of target molecules in a sample comprises: (i) contacting the sample comprising the target molecules with a plurality of first affinity agents having a binding affinity for the target molecules to produce a plurality of first complexes comprising a target molecule bound to a first affinity agent, wherein at least a portion of the plurality of first affinity agents are immobilized to a surface; (ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules; (iii) removing unbound second affinity agents and/or isolating the plurality of second complexes; (iv) optionally isolating at least a segment of each of the label molecules from the bound second affinity agents of the plurality of second complexes; (v) combining the segments of the label molecules with a known concentration of reference molecules; (vi) determining the ratio of detection events of label molecules relative to detection events of reference molecules; and (vii) determining the concentration of target molecules in the sample based at least in part on the ratio of detection events of label molecules relative to detection events of reference molecules.

In some embodiments, a method of determining the concentration of target molecules in a sample comprises: (i)(a) contacting the sample comprising the target molecules with a plurality of first affinity agents having a binding affinity for the target molecules to produce a plurality of first complexes comprising a target molecule bound to a first affinity agent, (i)(b) immobilizing at least a portion of the plurality of first affinity agents to a surface; (ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules; (iii) removing unbound second affinity agents and/or isolating the plurality of second complexes; (iv) optionally isolating at least a segment of each of the label molecules from the bound second affinity agents of the plurality of second complexes; (v) combining the isolated segments of the label molecules with a known concentration of reference molecules; (vi) determining the ratio of detection events of label molecules relative to detection events of reference molecules; and (vii) determining the concentration of target molecules in the sample based at least in part on the ratio of detection events of label molecules relative to detection events of reference molecules.

In some embodiments, a method of determining the concentration of labeled target molecules in a sample comprises: combining the sample containing labeled target molecules with a known concentration of reference molecules; determining the ratio of detection events of labeled target molecules relative to detection events of reference molecules; and determining the concentration of labeled target molecules in the sample based at least in part on the ratio of detection events of labeled target molecules relative to detection events of reference molecules.

In some embodiments, the sample is a biological sample. In some embodiments, the biological sample is a single cell, mammalian cell tissue, animal sample, fungal sample, or plant sample. In some embodiments, the biological sample is a blood sample, saliva sample, sputum sample, fecal sample, urine sample, buccal swab sample, amniotic sample, seminal sample, synovial sample, spinal sample, or pleural fluid sample.

In some embodiments, the target molecules are proteins, small molecules, or nucleic acids. In some embodiments, the nucleic acids are DNA and/or RNA molecules.

In some embodiments, the contacting of (i) and/or (ii) is performed at a temperature of 4-37° C., optionally 4-25° C. In some embodiments, the contacting of (i) and/or (ii) is performed for 5 minutes to 4 hours.

In some embodiments, the first affinity agents are antibodies or aptamers, and the target molecules are antigens, optionally wherein the antigens are proteins, peptides or polysaccharides. In some embodiments, the first affinity agents are antibodies or aptamers, the target molecules are proteins, and the antibodies or aptamers specifically bind to an epitope of the target molecules.

In some embodiments, at least a portion of the plurality of first affinity agents are immobilized to the surface of a solid-phase bead, microfluidic channel, nanoaperture, resin, matrix, membrane, polymer, plastic, metallic, or glass. In some embodiments, the solid-phase bead is a magnetic bead.

In some embodiments, the second affinity agents are antibodies.

In some embodiments, each of the label molecules (e.g., labeled target molecules) is linked to at least one fluorophore. In some embodiments, each of the label molecules (e.g., labeled target molecules) is linked to 2, 3, 4, or 5 distinct fluorophores. In some embodiments, each of the label molecules (e.g., labeled target molecules) is linked to 2, 3, 4, or 5 identical fluorophores.

In some embodiments, the method further comprises chemically linking at least one fluorophore to each of the label molecules (e.g., labeled target molecules) following (iv).

In some embodiments, each of the label molecules (e.g., labeled target molecules) comprises a chemical linker connected to at least one fluorophore. In some embodiments, each of the label molecules (e.g., labeled target molecules) comprises a biotin-streptavidin complex connected to at least one fluorophore. In some embodiments, each of the label molecules (e.g., labeled target molecules) comprises a biotin-streptavidin complex linked to a nucleic acid and at least one fluorophore. In some embodiments, each of the label molecules (e.g., labeled target molecules) is a label nucleic acid, wherein each of the second affinity agents is linked to a first strand of the label nucleic acid, and optionally wherein the first strand is 5-50 nucleobases in length.

In some embodiments, the first strand of the label nucleic acid is linked to a second affinity agent via a biotin-streptavidin complex. In some embodiments, the label nucleic acid is a single-stranded nucleic acid. In some embodiments, the single-stranded nucleic acid comprises a region that forms a hairpin loop. In some embodiments, the single-stranded nucleic acid is linked to at least one fluorophore.

In some embodiments, the label nucleic acid is a double-stranded nucleic acid comprising the first strand and a second strand comprising a region of complementarity to the first strand. In some embodiments, the second strand is linked to at least one fluorophore.

In some embodiments, the label nucleic acid comprises the first strand and a nucleic acid dumbbell, wherein a first region of the nucleic acid dumbbell is complementary to the first strand. In some embodiments, the nucleic acid dumbbell is linked to at least one fluorophore. In some embodiments, the label nucleic acid further comprises a second single-stranded nucleic acid that is complementary to a second region of the nucleic acid dumbbell. In some embodiments, the second single-stranded nucleic acid is linked to at least one fluorophore.

In some embodiments, the label nucleic acid comprises the first strand and a second single-stranded nucleic acid, wherein the first strand comprises a region that forms a hairpin loop. In some embodiments, the second single-stranded nucleic acid is linked to at least one fluorophore.

In some embodiments, (i) and (ii) occur simultaneously or in series. In some embodiments, (iii) comprises removing the unbound second affinity agents by washing the sample with a wash buffer. In some embodiments, a wash buffer is phosphate-buffered saline. In some embodiments, (iv) comprises isolating the at least a segment of each of the label molecules (e.g., labeled target molecules) from the bound second affinity agents by washing the sample with an elution buffer. In some embodiments, the elution buffer is a high-salt buffer. In some embodiments, (iv) comprises isolating the at least a segment of each of the label molecules (e.g., labeled target molecules) from the bound second affinity agents by altering the temperature (e.g., increasing the temperature) of the sample.

In some embodiments, the label nucleic acids are double-stranded label nucleic acids, wherein the second affinity agents are linked to a first strand of the double-stranded label nucleic acids, and wherein the isolated segments of the label nucleic acids are the second strands of the double-stranded label nucleic acids.

In some embodiments, (iv) comprises isolating the second strands of the double-stranded label nucleic acids by washing the sample with an excess of single-stranded nucleic acids that are complementary to the first strand of the double-stranded label nucleic acids.

In some embodiments, each of the reference molecules is linked to at least one fluorophore, wherein the fluorophore linked to a reference molecule is distinct from at least one fluorophore linked to a label molecule. In some embodiments, at least one fluorophore linked to a reference molecule and the at least one fluorophore linked to a label molecule (e.g., labeled target molecule) can be excited by the same excitation wavelength. In some embodiments, each of the reference molecules is a reference nucleic acid. In some embodiments, the reference nucleic acid is a single-stranded or double-stranded nucleic acid. In some embodiments, each of the reference molecules are immobilized to the same surface as the at least a portion of the plurality of first affinity agents. In some embodiments, each of the reference molecules are immobilized to a surface that is different from the surface to which the at least a portion of the plurality of first affinity agents are immobilized. In some embodiments, each of the reference molecules is linked to a first affinity agent. In some embodiments, each of the reference molecules is a complex comprising a single-stranded nucleic acid immobilized to a surface or linked to a first affinity agent and a dumbbell nucleic acid comprising a region of complementarity for the single-stranded nucleic acid. In some embodiments, the reference molecules are isolated during (iv).

In some embodiments, the label molecules (e.g., labeled target molecules) are label nucleic acids, the reference molecules are reference nucleic acids, and the label and reference nucleic acids are amplified during (iv), optionally using rolling circle amplification.

In some embodiments, the label molecules (e.g., labeled target molecules) (or a segment of the label molecules) and the known concentration of reference molecules are combined in a detection chip. In some embodiments, the detection chip comprises an ordered array of sample wells.

In some embodiments, the depth of each sample well is 50-500 nm, optionally about 300 nm. In some embodiments, the diameter of the interior base of each sample well is 50-250 nm, optionally 75-125 nm, further optionally about 100 nm.

In some embodiments, the interior base of each sample well is functionalized with a silane-containing compound. In some embodiments, the interior base of each sample well is functionalized with a biotin-streptavidin complex. In some embodiments, the interior base of each sample well is functionalized with positively-charged molecules. In some embodiments, the interior base of each sample well is functionalized with 40-300 positive charges per 1000 nm². In some embodiments, the positively-charged molecules are polylysine molecules. In some embodiments, the polylysine molecules comprise 10-200 lysine amino acids, optionally 20-100 lysine amino acids, further optionally 50 lysine amino acids. In some embodiments, the positively-charged molecules are positively-charged terminated silane molecules.

In some embodiments, the interior base of each sample well is functionalized with nucleic acids that are complementary to the label nucleic acids and/or reference nucleic acids.

In some embodiments, the ratio of label molecules (e.g., labeled target molecules) relative to reference molecules is determined using fluorescence measurements. In some embodiments, the ratio of label molecules (e.g., labeled target molecules) relative to reference molecules is determined using fluorescence measurements of label and reference molecules in the sample wells. In some embodiments, the ratio of label molecules (e.g., labeled target molecules) relative to reference molecules is determined in part based on the dwell time of label and reference molecules in the sample wells.

In some embodiments, the label and reference molecules are delivered to and maintained in the sample wells by electrostatic interactions with positively-charged molecules at the interior base of each sample well. In some embodiments, the label and reference molecules are delivered to and maintained in the sample wells by interactions with nucleic acids at the interior base of each sample well that are complementary to the label nucleic acids and/or reference nucleic acids. In some embodiments, the label and reference molecules are delivered to and maintained in the sample wells by gravity or a magnetic field. In some embodiments, the label and reference molecules are delivered to and maintained in the sample wells using a crowding reagent, optionally wherein the crowding reagent is a sugar molecule, methylcellulose, polyethylene glycol, dextran, ficoll, bovine serum albumin, or trehalose.

In some embodiments, the concentration of target molecules in the sample is determined using a standard curve derived from measurements of standard samples comprising known concentrations of label molecules (e.g., labeled target molecules) and reference molecules.

Some aspects of the disclosure provide a detection chip comprising an array of sample wells, wherein the interior base of each sample well is functionalized with positively-charged molecules.

In some embodiments, the array of sample wells is an ordered array. In some embodiments, the depth of each sample well is 50-500 nm, optionally about 300 nm. In some embodiments, the diameter of the interior base of each sample well is 50-250 nm, optionally 75-125 nm, further optionally 100 nm.

In some embodiments, the positively-charged molecules are attached to the interior base of each sample well using a silane-containing compound. In some embodiments, the positively-charged molecules are attached to the interior base of each sample well using a biotin-streptavidin complex. In some embodiments, the interior base of each sample well is functionalized with 40-300 positive charges per 1000 nm². In some embodiments, the positively-charged molecules are polylysine molecules. In some embodiments, the polylysine molecules comprise 10-200 lysine amino acids, optionally 20-100 lysine amino acids, further optionally 50 lysine amino acids. In some embodiments, the positively-charged molecules are positively-charged terminated silane molecules.

In some embodiments, the labeled target molecules or label molecules further comprise a molecular barcode.

Some aspects of the disclosure provide a method of determining the identity of a target molecule.

In some embodiments, a method of determining the identity of a target molecule comprises (i) contacting the target molecule with a first affinity agent having a binding affinity for the target molecule to produce a first complex comprising the target molecule bound to the first affinity agent, wherein the first affinity agent is immobilized to the surface of a solid-phase bead, and wherein a label molecule is attached to the surface of the solid-phase bead; (ii) contacting the first complex with a surface-immobilized second affinity agent having a binding affinity for the target molecule to produce a second complex comprising a second affinity agent bound to the first complex; (iii) optionally washing the second complex; (iv) isolating the label molecule; (v) contacting the isolated label molecule with a detection chip comprising a sample well, wherein a known molecule is attached to the sample well; and (vi) determining the identity of the label molecule using fluorescence, luminescence, and/or kinetic measurements, thereby identifying the target molecule.

In some embodiments, a method of determining the identity of a target molecule comprises (i) contacting the target molecule with a first affinity agent having a binding affinity for the target molecule to produce a first complex comprising the target molecule bound to the first affinity agent, wherein the first affinity agent is immobilized to the surface of a solid-phase bead; (ii) contacting the first complex with a second affinity agent having a binding affinity for the target molecule to produce a second complex comprising a second affinity agent bound to the first complex, wherein the second affinity agent is attached to a label molecule; (iii) optionally washing the second complex; (iv) isolating the label molecule; (v) contacting the isolated label molecule with a detection chip comprising a sample well, wherein a known molecule is attached to the sample well; and (vi) determining the identity of the label molecule using fluorescence, luminescence, and/or kinetic measurements, thereby identifying the target molecule.

In some embodiments, the label molecule is a label nucleic acid. In some embodiments, the known molecule is a known nucleic acid, optionally wherein the known nucleic acid is complementary to the label nucleic acid.

In some embodiments, the kinetic measurements comprise dwell time of the label molecule in the sample wells.

In some embodiments, the target molecule is a protein, small molecule, or nucleic acid. In some embodiments, the nucleic acid is DNA or RNA.

In some embodiments, the first affinity agents is an antibody or aptamer, and the target molecule is an antigen, optionally wherein the antigen is a protein, peptide or polysaccharide.

In some embodiments, the solid-phase bead is a plastic, polymer, glass or magnetic bead.

In some embodiments, the second affinity agent is an antibody.

In some embodiments, the label molecule is linked to at least one fluorophore.

In some embodiments, the detection chip comprises an ordered array of sample wells. The depth of each sample well may be 50-500 nm, optionally about 300 nm. The diameter of the interior base of each sample well may be 50-250 nm, optionally 75-125 nm, further optionally about 100 nm.

In some embodiments, the label molecule further comprises a molecular barcode.

### BRIEF DESCRIPTION OF DRAWINGS

FIG. 1 shows an example workflow of a method of the disclosure that utilizes single-stranded label and reference nucleic acids.

FIG. 2 shows an example workflow of a method of the disclosure that utilizes double-stranded label and reference nucleic acids.

FIG. 3 shows an example workflow of a method of the disclosure that utilizes dumbbell label and reference nucleic acids.

FIG. 4 shows example positionings of reference molecules relative to label molecules (left) and several embodiments of label molecules (right).

FIG. 5 shows example configurations of label and reference molecules.

FIG. 6 shows several examples of methods for delivering label and reference molecules to a sample well.

FIG. 7 shows an example of surface immobilization of sample wells.

FIG. 8 shows an example of the interior base of a detection chip functionalized with polylysine.

FIG. 9 shows an example of the interior base of a detection chip functionalized with positively-charged silane.

FIGS. 10A-10D show dynamic pulsing behavior using a detection chip as described herein. FIG. 10A shows a representative trace of an aperture. FIG. 10B shows a histogram of pulse counts in a 15 min period (pulse rate). FIG. 10C shows cluster separation of two different nucleic acids labeled with fluorophores with a signature difference in bin-ratio for pulses with a pulse duration of >0.3. FIG. 10D shows a histogram of pulse duration.

FIGS. 11A-11B show example methods of modifying an antibody with a cycooctyne-containing molecule (FIG. 11A) and a biotin-streptavidin complex (FIG. 11B).

FIG. 12A-12D shows example strategies for eluting a nucleic acid molecule (e.g., a label molecule) from a complex comprising a first affinity agent and a second affinity agent (e.g., a sandwiched antibody-antigen complex).

FIG. 13 shows an example trace demonstrating the initiation of pulsing upon addition of the labeled dsDNA due to reversible binding of the dsDNA to a PLL-functionalized surface at the bottom of a nanoaperture.

FIG. 14 shows a measure of the relative signal collected in the lifetime-sensitive time bins of a complementary metal-oxide-semiconductor (CMOS) sensor that corresponds to fluorescence lifetimes of dyes (binratio) as collected during a detection run of a sample containing 25 pM each of a Cy3 and Atto-Rho6G labelled dsDNA.

FIGS. 15A-15B provide example workflows of methods of the disclosure that utilize.

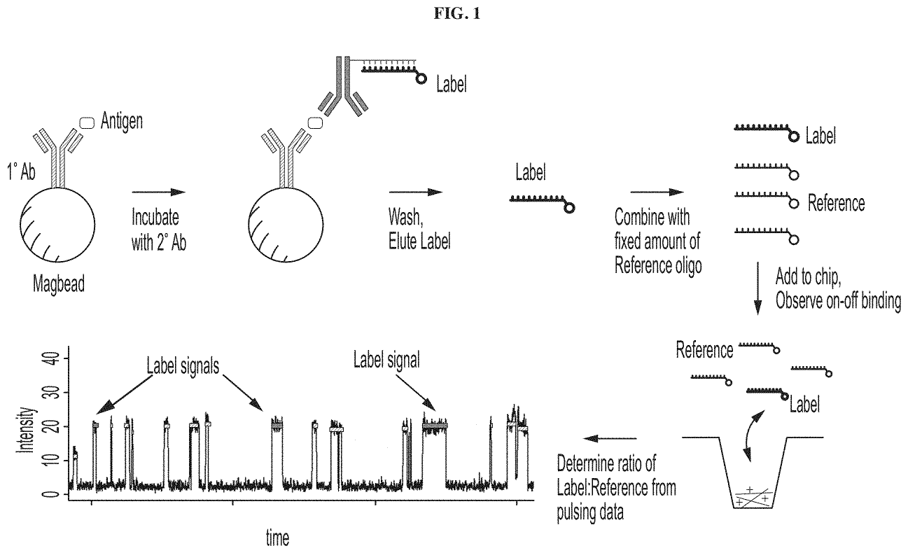

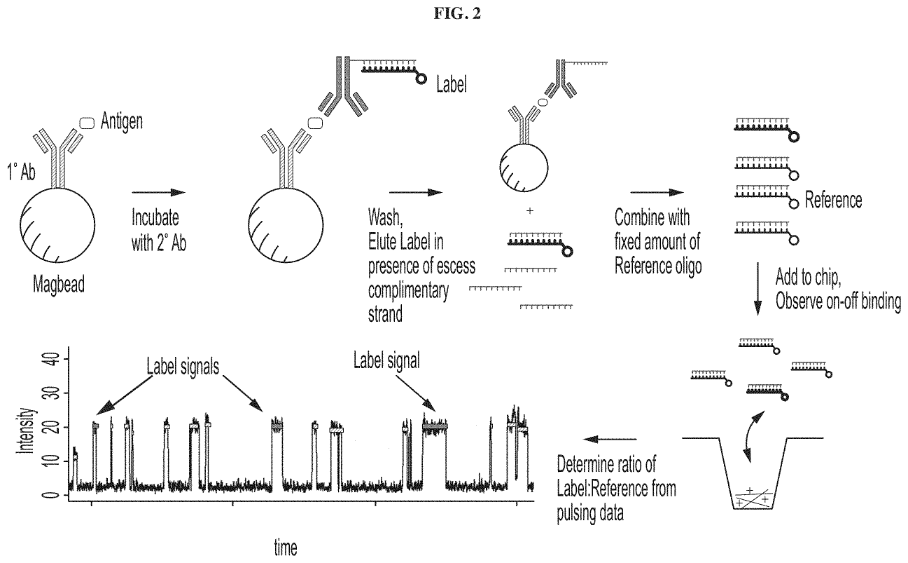

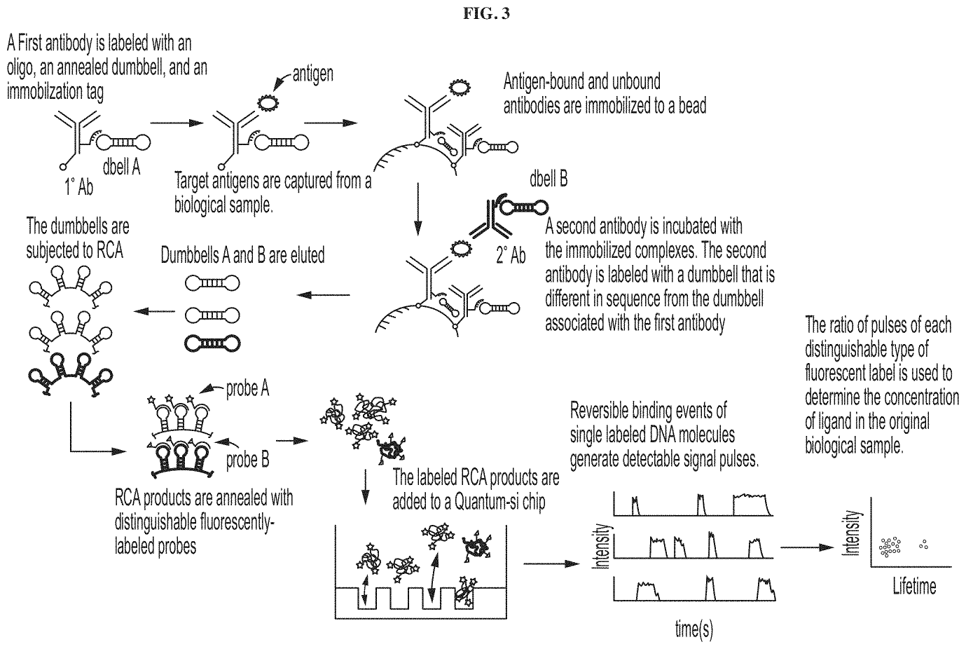

### DETAILED DESCRIPTION

In some aspects, the disclosure provides methods of biosensor detection (e.g., accurate determination of amount, e.g., concentration, of a target molecule). In some embodiments, a method of biosensor detection (e.g., determining the concentration of labeled target molecules in a sample) comprises combining the sample containing labeled target molecules with a known concentration of reference molecules; determining the ratio of detection events of labeled target molecules relative to detection events of reference molecules; and determining the concentration of labeled target molecules in the sample based at least in part on the ratio of detection events of labeled target molecules relative to detection events of reference molecules. In some embodiments, a method of biosensor detection comprises determining the identity of a target molecule.

In some embodiments, a method of biosensor detection (e.g., determining the concentration of target molecules in a sample) comprises:

(i) contacting the sample comprising the target molecules with a plurality of first affinity agents having a binding affinity for the target molecules to produce a plurality of first complexes comprising a target molecule bound to a first affinity agent, wherein at least a portion of the plurality of first affinity agents are immobilized to a surface;

(ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules;

(iii) removing unbound second affinity agents and/or isolating the plurality of second complexes;

(iv) optionally isolating at least a segment of each of the label molecules from the bound second affinity agents of the plurality of second complexes;

(v) combining the segments of the label molecules with a known concentration of reference molecules;

(vi) determining the ratio of detection events of label molecules relative to detection events of reference molecules; and

(vii) determining the concentration of target molecules in the sample based at least in part on the ratio of detection events of label molecules relative to detection events of reference molecules.

In some embodiments, step (i) comprises: (i)(a) contacting the sample comprising the target molecules with a plurality of first affinity agents having a binding affinity for the target molecules to produce a plurality of first complexes comprising a target molecule bound to a first affinity agent, and (i)(b) immobilizing at least a portion of the plurality of first affinity agents to a surface.

### Target Molecules

A target molecule may be any protein, small molecule, or nucleic acid. In some embodiments, a target molecule is a naturally occurring molecule. In some embodiments, a target molecule is a synthetic molecule. In some embodiments, a target molecule is derived from or obtained from a biological sample. A target molecule may be an antigens. In some embodiments, a target molecule is an antigen, wherein the antigen is a protein, peptide or polysaccharide.

A biological sample may be a single cell, mammalian cell tissue, animal sample, fungal sample, or plant sample. In some embodiments, a biological sample is a blood sample, saliva sample, sputum sample, fecal sample, urine sample, buccal swab sample, amniotic sample, seminal sample, synovial sample, spinal sample, or pleural fluid sample.

In some embodiments, a sample may be a purified sample, a cell lysate, a single-cell, a population of cells, or a tissue. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram negative bacterial cells. In some embodiments, a sample is a purified sample of nucleic acids or proteins that have been previously extracted via user-developed methods from metagenomic samples or environmental samples. A blood sample may be a freshly drawn blood sample from a subject (e.g., a human subject) or a dried blood sample (e.g., preserved on solid media (e.g. Guthrie cards)). A blood sample may comprise whole blood, serum, plasma, red blood cells, and/or white blood cells.

In some embodiments, a sample (e.g., a sample comprising cells or tissue), may be prepared, e.g., lysed (e.g., disrupted, degraded and/or otherwise digested) in a process known a skilled person in the art. In some embodiments, a sample to be prepared, e.g., lysed, comprises cultured cells, tissue samples from biopsies (e.g., tumor biopsies from a cancer patient, e.g., a human cancer patient), or any other clinical sample. In some embodiments, a sample comprising cells or tissue is lysed using any one of known physical or chemical methodologies to release a target molecule (e.g., a target nucleic acid or a target protein) from said cells or tissues. In some embodiments, a sample may be lysed using an electrolytic method, an enzymatic method, a detergent-based method, and/or mechanical homogenization. In some embodiments, a sample (e.g., complex tissues, gram positive or gram negative bacteria) may require multiple lysis methods performed in series. In some embodiments, if a sample does not comprise cells or tissue (e.g., a sample comprising purified nucleic acids), a lysis step may be omitted. In some embodiments, lysis of a sample is performed to isolate target nucleic acid(s). In some embodiments, lysis of a sample is performed to isolate target protein(s). In some embodiments, a lysis method further includes use of a mill to grind a sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

### Affinity Agents

A first affinity agent as described herein is a molecule (e.g., an antibody or an aptamer) that has a binding affinity for a target molecule. In some embodiments, a first affinity agent has a binding affinity for a target molecule of between 1×10⁻³ M to 1×10⁻⁴ M, ×10⁻⁴ M to 1×10⁻⁵ M, 1×10⁻⁵ M to 1×10⁻⁶ M, 1×10⁻⁶ M to 1×10⁻⁷ M, 1×10⁻⁷ M to 1×10⁻⁸ M, 1×10⁻⁸M to 1×10⁻⁹ M, 1×10⁻⁹ M to 1×10⁻⁹ M, 1×10⁻⁹ M to 1×10⁻¹⁰ M, or 1×10⁻¹² M to 1×10⁻¹² M. In some embodiments, a first affinity agent binds specifically to a particular epitope of a target molecule (e.g., a target protein). In some embodiments, a first affinity agent is a primary antibody.

A first affinity agent may be immobilized to a surface. In some embodiments, a first affinity agent is immobilized to a surface before being contacted with (e.g., binding to) its cognate target molecule for which it has a binding affinity. In other embodiments, a first affinity agent is immobilized to a surface after it has been contacted with (e.g., bound to) its cognate target molecule for which it has a binding affinity, e.g., such that a complex has been formed between the first affinity agent and the target molecule. In some embodiments, a first affinity agent is immobilized to a solid-phase bead (e.g., a magnetic bead, e.g., a Dynabead), microfluidic channel, nanoaperture, resin, matrix, membrane, polymer, plastic, metallic, or glass.

A second affinity agent as described herein is generally a molecule (e.g., an antibody or an aptamer) that has a binding affinity for a complex comprising a first affinity agent bound to its cognate target molecule. In some embodiments, a second affinity agent has a binding affinity for a complex comprising a first affinity agent bound to its cognate target molecule of between 1×10⁻³ M to 1×10⁻⁴ M, 1×10⁻⁴ M to 1×10⁻⁵ M, 1×10⁻⁵ M to 1×10⁻⁶ M, 1×10⁻⁶ M to 1×10⁻⁷ M, 1×10⁻⁷ M to 1×10⁻⁸ M, 1×10⁻⁸ M to 1×10⁻⁹ M, 1×10⁻⁹ M to 1×10⁻⁹ M, 1×10⁻⁹ M to 1×10⁻¹⁰ M, or 1×10⁻¹⁰ M to 1×10⁻¹² M.

### Label and Reference Molecules

In some embodiments, a label molecule is a molecule that is linked to a second affinity agent. In some embodiments of the methods described herein, a label molecule is used to represent the total quantity or amount (e.g., concentration) of a target molecule within a sample. A label molecule may be detected to provide an accurate determination of the total quantity or amount (e.g., concentration) of a target molecule within a sample. Detection of a label molecule may be performed using sequencing technologies (e.g., protein sequencing or nucleic acid sequencing) or fluorescence measurements.

A label molecule may comprise a nucleic acid. In some embodiments, a label molecule is a label nucleic acid. In some embodiments, a label molecule comprises a biotin-streptavidin complex linked to a nucleic acid and at least one fluorophore. In some embodiments, a label molecules is a label nucleic acid comprising two complementary strands, wherein the first strand is linked to a second affinity agent

In some embodiments, a label nucleic acid comprises 5-200, 5-150, 5-100, 5-50, 5-25, 10-200, 10-100, 10-50, 25-200, 25-100, 25-50, 50-200, 50-100, or 100-200 nucleotides in length. A label nucleic acid may be a single-stranded or double-stranded nucleic acid. A label nucleic acid may comprise a secondary or tertiary structural element. In some embodiments, a label nucleic acid comprises a region that forms a hairpin loop. In some embodiments, a label nucleic acid is a dumbbell nucleic acid. In some embodiments, a label nucleic acid is a double-stranded nucleic acid comprising a first strand and a second strand comprising a region of complementarity to the first strand.

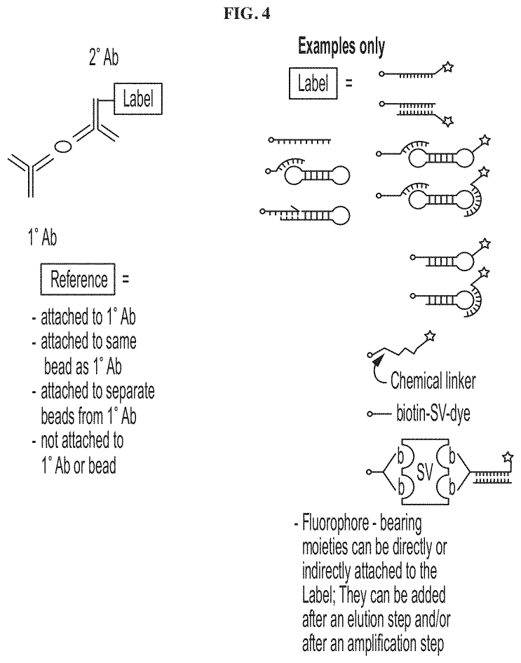

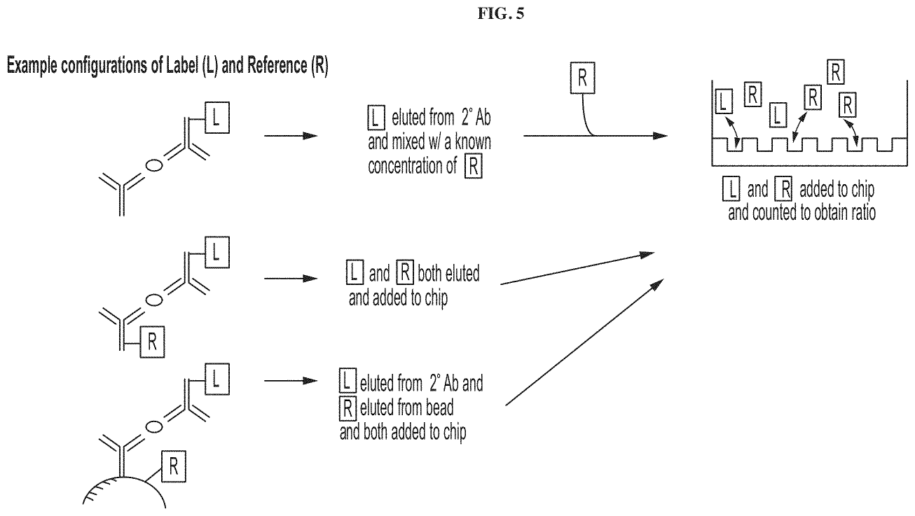

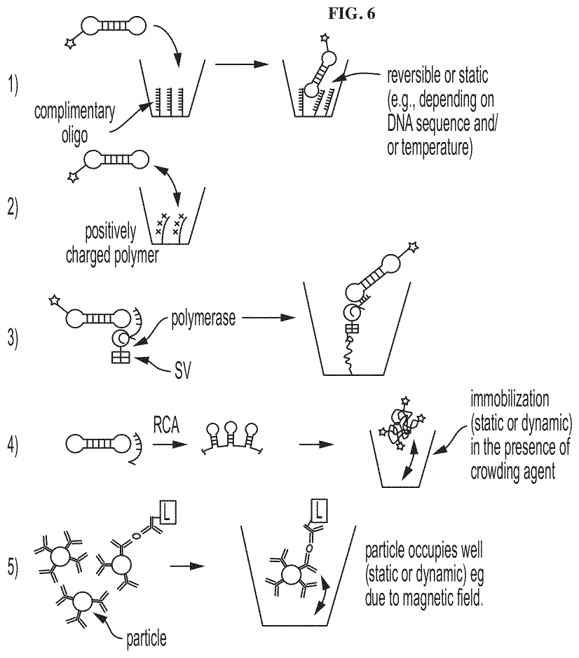

A label molecule may be as shown in FIG. 4. For example, in some embodiments, a label nucleic acid comprises a first strand and a nucleic acid dumbbell, wherein a first region of the nucleic acid dumbbell is complementary to the first strand. In some embodiments, a label nucleic acid comprising a first strand and a nucleic acid dumbbell the label nucleic acid further comprises a second single-stranded nucleic acid that is complementary to a second region of the nucleic acid dumbbell.

In some embodiments, a label molecule is linked to at least one fluorophore. In some embodiments, a label molecule is linked to 1, 2, 3, 4, 5, 6, 7, 8, 9, or 10 fluorophores. In some embodiments, a label molecule is linked to 1, 2, 3, 4, 5, 6, 7, 8, 9, or 10 distinct fluorophores (e.g., each fluorophore is excited by a distinct excitation wavelength). In some embodiments, a label molecule is linked to 1, 2, 3, 4, 5, 6, 7, 8, 9, or 10 identical fluorophores (e.g., each fluorophore is excited by the same excitation wavelength).

Fluorophores may be linked (e.g., chemically linked) to a label molecule before or after the label molecule has been linked to a second affinity agent. In some embodiments, a fluorophore is linked to a label molecule via a carbon-based chemical linkage. In some embodiments, a fluorophore is linked to a label molecule via a biotin-streptavidin complex.

A reference molecule is a molecule that, in some embodiments, is used as an internal standard in a sample (e.g., added to the sample by the operator in a defined, known quantity) In some embodiments, the reference molecules is a reference nucleic acid (e.g., a single-stranded or double-stranded nucleic acid). A reference molecule may be immobilized to a surface (e.g., the same surface as the first affinity agent). In some embodiments, a reference molecule is linked to a first affinity agent. In some embodiments, a reference molecule is not immobilized to any surface.

In some embodiments, a reference molecule is a complex comprising a single-stranded nucleic acid immobilized to a surface or linked to a first affinity agent and a dumbbell nucleic acid comprising a region of complementarity for the single-stranded nucleic acid.

### Determination of Ratio of Detection Events of Label and Reference Molecules

In methods of the disclosure, the amount (e.g., concentration) of a target molecule is determined based on the ratio of detection events of label molecules relative to detection events of reference molecules, wherein the detection events of the label molecules represent the presence of the target molecule. In some embodiments, the detection event may be determined using sequencing (e.g., protein sequencing or nucleic acid sequencing) or fluorescence measurements. In methods of the disclosure, the amount (e.g., concentration) of reference molecules is known when determining the ratio of detection events of label molecules relative to detection events of reference molecules. In some embodiments, the ratio of detection events of label molecules relative to detection events of reference molecules in a sample (e.g., a biological sample) is plotted against a standard curve (e.g., a standard curve generated using mixtures of known quantities of the label molecule and the reference molecule). In some embodiments, a standard curve is derived from measurements of standard samples comprising known concentrations of label molecules and reference molecules.

In some embodiments involving fluorescence measurements, isolated segments of the label molecules and the known concentration of reference molecules are combined in a detection chip in order to determination the ratio of detection events of label molecules relative to detection events of reference molecules. In some embodiments involving sequencing measurements, isolated segments of the label molecules and the known concentration of reference molecules are combined into a sample to be sequenced in order to determination the ratio of detection events of label molecules relative to detection events of reference molecules.

In some embodiments, the ratio of detection events of label molecules relative to detection events of reference molecules is determined using fluorescence measurements of label and reference molecules in sample wells of a detection chip.

The ratio of label molecules relative to reference molecules may be determined in part based on the dwell time of label molecules and reference molecules in the sample wells of a detection chip. The dwell time of label and reference molecules in the detection chip may, in some embodiments, be directly correlated with the concentration of label and reference molecules in the sample (and in the sample wells of the detection chip). In some embodiments, the dwell time of molecules can be slowed down, e.g., to improve signal confidence and to lower detection limits. In some embodiments, label and reference molecules are delivered to and maintained in the sample wells by electrostatic interactions with positively-charged molecules (e.g., polylysine) at the interior base of each sample well. In some embodiments, the label and reference nucleic acids are delivered to and maintained in the sample wells by interactions with nucleic acids at the interior base of each sample well that are complementary to the label nucleic acids and/or reference nucleic acids. In some embodiments, the label and reference proteins are delivered to and maintained in the sample wells by interactions with antibodies at the interior base of each sample well that have a binding affinity for the label nucleic acids and/or reference nucleic acids. In some embodiments, the label and reference molecules are delivered to and maintained in the sample wells by gravity or a magnetic field (e.g., if the label and reference molecules remain linked to a solid surface, e.g., of a solid-phase bead, e.g., a magnetic bead). In some embodiments, the label and reference molecules are delivered to and maintained in the sample wells using a crowding reagent. A crowding reagent may include, for example, a sugar molecule (e.g., sucrose or trehalose), methylcellulose, polyethylene glycol, dextran, ficoll, or a protein such as bovine serum albumin.

Measurement of dwell time may be performed using any suitable buffer. In some embodiments, measurements of dwell time are performed using NaCl 200-350 mM, KCl 10-30 mM, Na2HPO4 3 mM, KHPO4 1 mM, 4-Nitrobenzoic acid 5 mM, D-glucose 50 mM, 0.1% Tween-20, pH=7.5).

In some embodiments, fluorescence measurements (e.g., dwell time) are analyzed using pulse calling. In some embodiments, the reversible binding of label and reference molecules to the positively-charged surface of a detection chip will result in transient pulses, which can then be called and filtered based on their properties (e.g., pulse durations, single-frame pulses). In some embodiments, pulses with low single-frame pulse are discarded. Then, in some embodiments, the operator may generate statistics of qualified pulses, e.g., histogram of pulses/aperture and pulse durations, and/or generate histogram of bin-ratios to show label and reference cluster separation, based on which quantification of the molecules (e.g., the fluorophores of the molecules) can be achieved.

### Method Conditions

The method may be performed at any reasonable temperature. For example, any step of the method (e.g., contacting the first affinity agent with the sample) may be performed at a temperature of 4-40° C., 4-37° C., 4-30° C., 4-25° C., 4-15° C., 4-10° C., 10-40° C., 15-37° C., 15-25° C., or room temperature. Similarly, any step of the method may be performed for any reasonable period of time. For example, any step of the method (e.g., contacting the first affinity agent with the sample) may be performed for 5-60 minutes, 5-300 minutes, 5-200 minutes, 5-100 minutes, 30-180 minutes, 1-4 hours, 1-3 hours, or 1-2 hours.

In some embodiments, a sample is contacted with the first affinity agent before being contacted with the second affinity agent. In other embodiments, a sample is simultaneously contacted with the first affinity agent and the second affinity agent.

Removing unbound second affinity agents from a sample is performed by washing the sample with a wash buffer. A wash buffer may be a high-salt wash buffer, low-salt wash buffer, or phosphate-buffered saline. Isolating second complexes comprising a first affinity agent, second affinity agent, and label molecule (e.g., second complexes linked to a surface) may be performed by filtering the sample or manually removing the surface (e.g., beads) to which the second complexes are linked.

Isolating the label molecules from the bound second affinity agents may be performed by washing the sample with an elution buffer. An elution buffer may be a high-salt buffer, a buffer with a pH distinct from the sample, or any elution buffer known to a skilled person in the art. In some embodiments the label molecules are isolated from the bound second affinity agents by altering the temperature (e.g., increasing the temperature) of the sample.

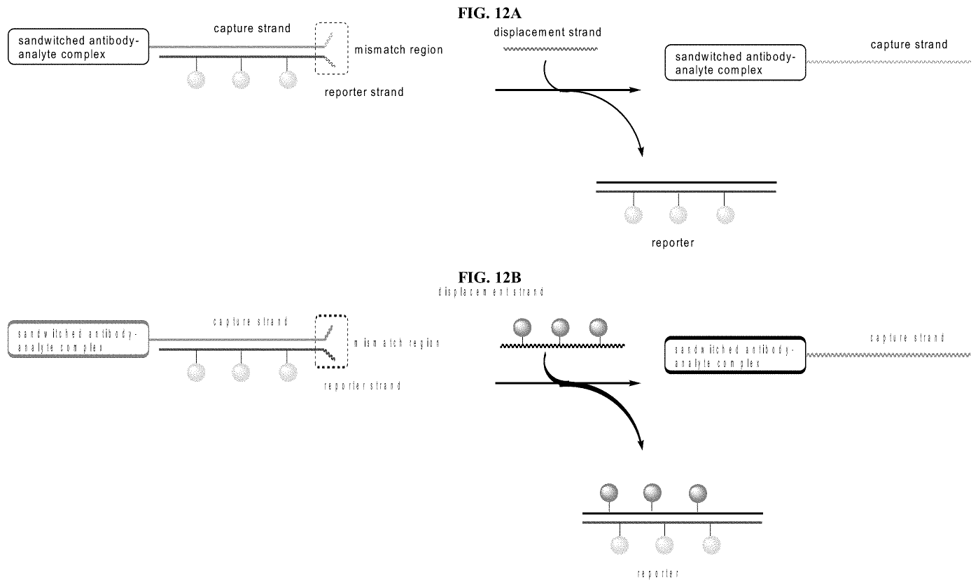

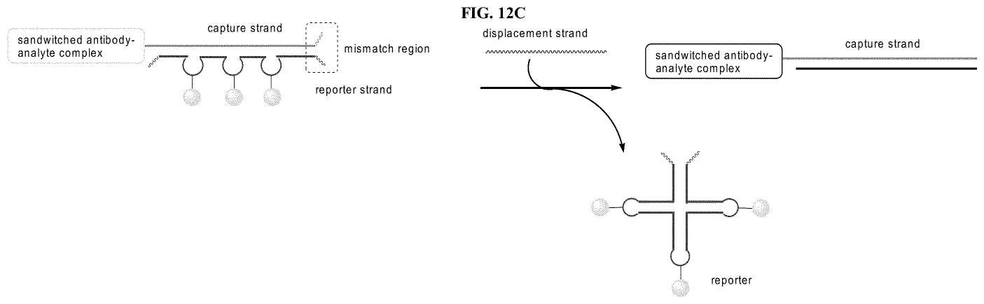

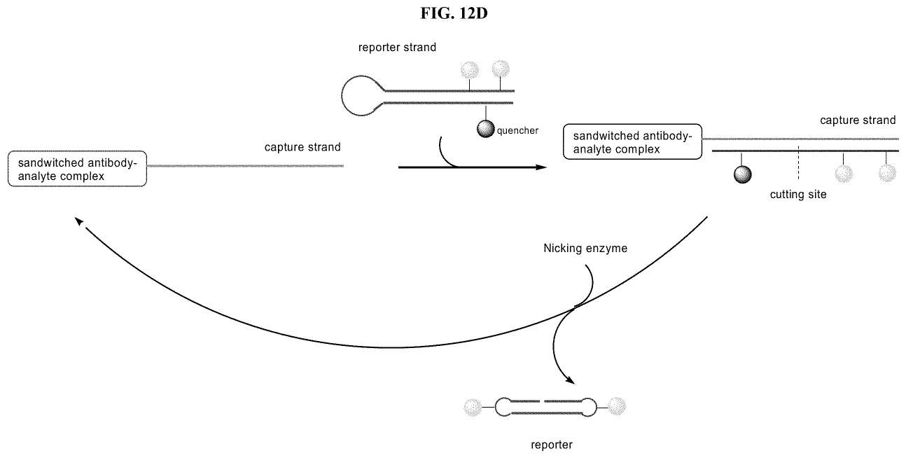

In some embodiments, a label nucleic acid is isolated using a process as demonstrated in FIGS. 12A-12D. FIGS. 12A-12B show that a label nucleic acid (i.e., reporter strand) can be isolated by washing the sample with an excess of single-stranded nucleic acids (i.e., displacement strand) that are complementary to the reporter strand of the double-stranded label nucleic acid. FIG. 12C shows that a label nucleic acid (i.e., reporter strand) can be isolated by washing the sample with an excess of single-stranded nucleic acids (i.e., displacement strand) that are complementary to the first strand (i.e., capture strand) of the double-stranded label nucleic acid. FIG. 12D shows that a label nucleic acid (i.e., reporter) can be isolated by contacting the sample with a nicking enzyme that cuts the label nucleic acid.

In some embodiments, if the reference molecule is linked to a surface or otherwise present in the sample prior to isolation of the label molecule, then the reference molecule may be isolated simultaneously with the label molecule. In some embodiments, the label and reference nucleic acids are amplified during or after being isolated from the sample. Nucleic acids may be amplified using any known amplification technique (e.g., rolling circle amplification)

### Cartridges or Detection Chips

In another aspect, cartridge or detection chips are provided. In some embodiments, a detection chip comprises an array of sample wells (e.g., an ordered array of sample wells). In some embodiments, a detection chip enables collection of fluorescence measurements. A detection chip may be functionalized with positively-charged molecules, e.g., at the interior base of each sample well (i.e., bottom of the sample well).

In some embodiments, the depth of a sample well in a detection chip is 50-500 nm, 25-250 nm, 50-400 nm, 50-300 nm, 50-200 nm, 50-150 nm, 50-100 nm, 75-150 nm, 100-250 nm, 100-300 nm, 250-500 nm, or 250-350 nm. In some embodiments, the depth of a sample well in a detection chip is about 50 nm, 75 nm, 100 nm, 125 nm, 150 nm, 175 nm, 200 nm, 250 nm, 300 nm, 350 nm, 400 nm, 450 nm, or 500 nm. In some embodiments, the diameter of the interior base of each sample well is 50-250 nm, 50-200 nm, 50-150 nm, 50-100 nm, 75-150 nm, 75-200 nm, 100-150 nm, 100-200 nm, or 150-250 nm. In some embodiments, the diameter of the interior base of each sample well is about 50 nm, 75 nm, 100 nm, 125 nm, 150 nm, 175 nm, 200 nm, or 250 nm.

In some embodiments, a detection chip comprises positively-charged molecules attached to the interior base of a sample well. In some embodiments, the positively-charged molecules are attached using a silane-containing compound or a biotin-streptavidin complex. In some embodiments, the interior base of a sample well is functionalized with 20-500, 30-400, 50-350, 50-300, 50-250, 50-200, 50-100, 100-400, or 100-200 positive charges per 1000 nm².

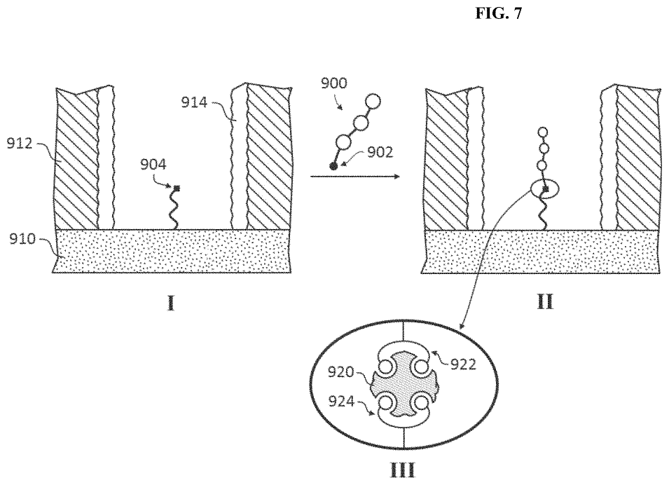

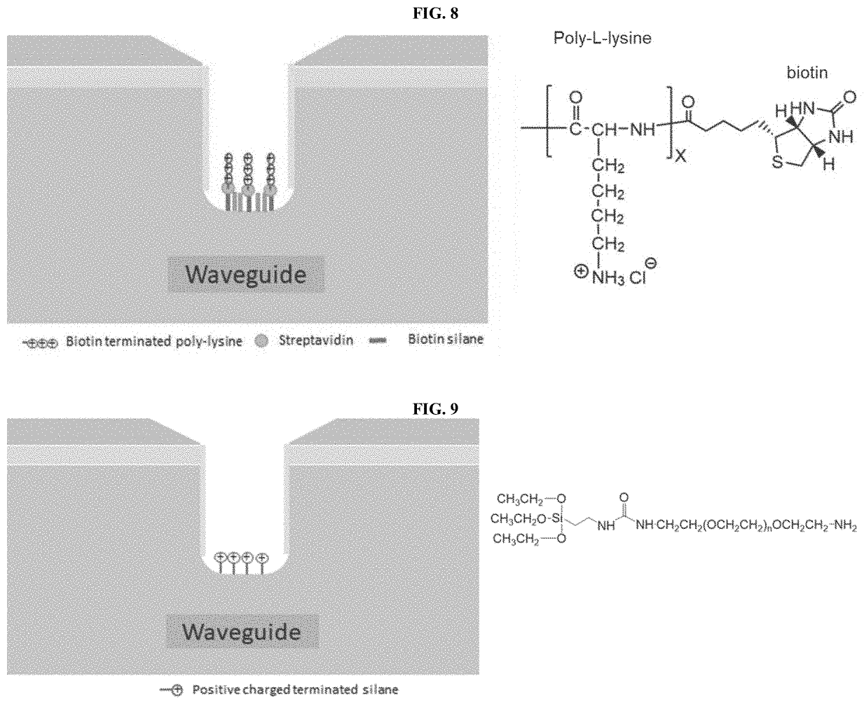

In some embodiments, the positively-charged molecules are polylysine molecules. The polylysine molecules may comprise a linear or branched chain of lysine amino acids. In some embodiments, the polylysine molecules comprise 10-200, 10-150, 10-100, 25-200, 25-150, 25-100, 20-100, 20-75, 25-50, or 50-100 lysine amino acids. In some embodiments, the polylysine molecules comprise about 10, 25, 50, 75, 100, 125, 150, 175, 200, or 225 lysine amino acids. In some embodiments, the polylysine molecules are as shown in FIG. 8.

In some embodiments, the positively-charged molecules are positively-charged terminated silane molecules. In some embodiments, the positively-charged terminated silane molecules are as shown in FIG. 9.

In some embodiments, a cartridge or detection chip comprises a base layer having a surface comprising channels, and at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel.

In some embodiments, a cartridge or detection chip comprises a base layer. In some embodiments, a base layer has a surface comprising one or more channels. As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring again to FIG. 24, in some embodiments, at least a portion of at least some of channels 102 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in certain embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) may have an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. In some embodiments, a portion along a length of a channel may have both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the cartridge or detection chip can allow for control of which portions of the channel are capable of undergoing pump action upon engagement with the apparatus.

The inclusion of such “deep sections” as second portions of at least some of the channels of the cartridge or detection chip may contribute to any of a variety of potential benefits. For example, such deep sections may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge or detection chip registration. The inclusion of deep sections may, in some cases, reduce or eliminate variations in pump volume associated with such error.

As used herein, an average diameter of a substantially triangular portion of a channel may be measured as an average over the z-axis from the vertex of the substantially triangular portion to the surface of the channel.

### Devices and Modules

Devices or modules including apparatuses, cartridge or detection chips (e.g., comprising channels (e.g., microfluidic channels)), and/or pumps (e.g., peristaltic pumps) for use in a method as described herein are generally provided. Devices can be used in accordance with the instant disclosure to promote accurate determination of the amount (e.g., concentration) of a target molecule from a biological sample. Devices and related methods may be used for performing chemical and/or biological reactions, including reactions for nucleic acid and/or protein processing in accordance with any steps of the biosensor detection methods described elsewhere herein.

A device of the disclosure may, in some embodiments, perform any number of the following steps: combining the sample containing labeled target molecules with a known concentration of reference molecules; determining the ratio of detection events of labeled target molecules relative to detection events of reference molecules; and determining the concentration of labeled target molecules in the sample based at least in part on the ratio of detection events of labeled target molecules relative to detection events of reference molecules.

A device of the disclosure may, in some embodiments, perform any number of the following steps:

(i) contacting the sample comprising the target molecules with a plurality of first affinity agents having a binding affinity for the target molecules to produce a plurality of first complexes comprising a target molecule bound to a first affinity agent, wherein at least a portion of the plurality of first affinity agents are immobilized to a surface;

(ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules;

(iii) removing unbound second affinity agents and/or isolating the plurality of second complexes;

(iv) optionally isolating at least a segment of each of the label molecules from the bound second affinity agents of the plurality of second complexes;

(v) combining the segments of the label molecules with a known concentration of reference molecules;

(vi) determining the ratio of detection events of label molecules relative to detection events of reference molecules; and

(vii) determining the concentration of target molecules in the sample based at least in part on the ratio of detection events of label molecules relative to detection events of reference molecules.

In some embodiments, step (i) comprises: (i)(a) contacting the sample comprising the target molecules with a plurality of first affinity agents having a binding affinity for the target molecules to produce a plurality of first complexes comprising a target molecule bound to a first affinity agent, and (i)(b) immobilizing at least a portion of the plurality of first affinity agents to a surface.

In some embodiments, a device of the disclosure performs all of steps (i)-(vii). In some embodiments, a device of the disclosure performs step (i) and optionally performs any number of steps (ii)-(vii). In some embodiments, a device of the disclosure performs step (ii) and optionally performs any number of steps (i) and/or (iii)-(vii). In some embodiments, a device of the disclosure performs step (iii) and optionally performs any number of steps (i), (ii), and/or (iv)-(vii). In some embodiments, a device of the disclosure performs step (iv) and optionally performs any number of steps (i)-(iii) and/or (v)-(vii). In some embodiments, a device of the disclosure performs step (v) and optionally performs any number of steps (i)-(iv), (vi), or (vii). In some embodiments, a device of the disclosure performs step (vi) and optionally performs any number of steps (i)-(v), (vii), and/or (viii). In some embodiments, a device of the disclosure performs step (vii) and optionally performs any number of steps (i)-(vi), and/or (viii). In some embodiments, a device of the disclosure performs step (viii) and optionally performs any number of steps (i)-(vii). The order of steps can be altered as necessary for an experiment. In some embodiments, any one of the steps is interspersed with manual steps. This flexibility enables the user to address multiple sample types and detection platforms.

In some embodiments, a device of the disclosure is positioned to deliver or transfer to a detection module or device a label molecule and/or a reference molecule. In some embodiments, a device of the disclosure is connected directly to (e.g., physically attached to) or indirectly to a detection device or module.

In some embodiments, a cartridge or detection chip comprises one or more reservoirs or reaction vessels configured to receive a fluid and/or contain one or more reagents used in a biosensor method. In some embodiments, a cartridge or detection chip comprises one or more channels (e.g., microfluidic channels) configured to contain and/or transport a fluid (e.g., a fluid comprising one or more reagents) used in a biosensor method. Reagents include buffers, enzymatic reagents, polymer matrices, capture reagents, size-specific selection reagents, sequence-specific selection reagents, and/or purification reagents. Additional reagents for use in a biosensor method are known to a skilled person in the art.

In some embodiments, a cartridge or detection chip includes one or more stored reagents (e.g., of a liquid or lyophilized form suitable for reconstitution to a liquid form). The stored reagents of a cartridge or detection chip include reagents suitable for carrying out a desired process and/or reagents suitable for processing a desired sample type. In some embodiments, a cartridge or detection chip is a single-use cartridge or detection chip (e.g., a disposable cartridge or detection chip) or a multiple-use cartridge or detection chip (e.g., a reusable cartridge or detection chip). In some embodiments, a cartridge or detection chip is configured to receive a user-supplied sample. The user-supplied sample may be added to the cartridge or detection chip before or after the cartridge or detection chip is received by the device, e.g., manually by the user or in an automated process.

Devices in accordance with the instant disclosure generally contain mechanical and electronic and/or optical components which can be used to operate a cartridge or detection chip as described herein. In some embodiments, the device components operate to achieve and maintain specific temperatures on a cartridge or detection chip or on specific regions of the cartridge or detection chip. In some embodiments, the device components operate to apply specific voltages for specific time durations to electrodes of a cartridge or detection chip. In some embodiments, the device components operate to move liquids to, from, or between reservoirs and/or reaction vessels of a cartridge or detection chip. In some embodiments, the device components operate to move liquids through channel(s) of a cartridge or detection chip, e.g., to, from, or between reservoirs and/or reaction vessels of a cartridge or detection chip. In some embodiments, the device components move liquids via a peristaltic pumping mechanism (e.g., apparatus) that interacts with an elastomeric, reagent-specific reservoir or reaction vessel of a cartridge or detection chip. In some embodiments, the device components move liquids via a peristaltic pumping mechanism (e.g., apparatus) that is configured to interact with an elastomeric component (e.g., surface layer comprising an elastomer) associated with a channel of a cartridge or detection chip to pump fluid through the channel. Device components can include computer resources, for example, to drive a user interface where sample information can be entered, specific processes can be selected, and run results can be reported.

In some embodiments, devices or modules (e.g., sample preparation devices; sequencing devices; combined sample preparation and sequencing devices) are configured to transport small volume(s) of fluid precisely with a well-defined fluid flow resolution, and with a well-defined flow rate in some cases. In some embodiments, devices or modules are configured to transport fluid at a flow rate of greater than or equal to 0.1 μL/s, greater than or equal to 0.5 μL/s, greater than or equal to 1 μL/s, greater than or equal to 2 μL/s, greater than or equal to 5 μL/s, or higher. In some embodiments, devices or modules herein are configured to transport fluid at a flow rate of less than or equal to 100 μL/s, less than or equal to 75 μL/s, less than or equal to 50 μL/s, less than or equal to 30 μL/s, less than or equal to 20 μL/s, less than or equal to 15 μL/s, or less. Combinations of these ranges are possible. For example, in some embodiments, devices or modules herein are configured to transport fluid at a flow rate of greater than or equal to 0.1 μL/s and less than or equal to 100 μL/s, or greater than or equal to 5 μL/s and less than or equal to 15 μL/s. For example, in certain embodiments, systems, devices, and modules herein have a fluid flow resolution on the order of tens of microliters or hundreds of microliters. Further description of fluid flow resolution is described elsewhere herein. In certain embodiments, systems, devices, and modules are configured to transport small volumes of fluid through at least a portion of a cartridge or detection chip.

In some embodiments, a cartridge or detection chip is capable of handling small-volume fluids (e.g., 1-10 μL, 2-10 μL, 4-10 μL, 5-10 μL, 1-8 μL, or 1-6 μL fluid). In some embodiments, the sequencing cartridge or detection chip is physically embedded or associated with a device of the disclosure (e.g., to allow for a prepared sample to be delivered to a reaction mixture for sequencing. In some embodiments, a sequencing cartridge or detection chip that is physically embedded or associated with a device of the disclosure comprises microfluidic channels that have fluid interfaces in the form of face sealing gaskets or conical press fits (e.g., Luer fittings). In some embodiments, fluid interfaces can then be broken after delivery of the prepared sample in order to physically separate the sequencing cartridge or detection chip from the device of the disclosure.

The following non-limiting example is meant to illustrate aspects of the devices, methods, and compositions described herein. The use of a device of the disclosure in accordance with the instant disclosure may proceed with one or more of the following described steps. A user may open the lid of the device and insert a cartridge or detection chip that supports the desired process. The user may then add a sample, which may be combined with a specific lysis solution, to a sample port on the cartridge or detection chip. The user may then close the device lid, enter any sample specific information via a touch screen interface on the device, select any process specific parameters (e.g., range of desired size selection, desired degree of homology for target molecule capture, etc.), and initiate the biosensor method run. Following the run, the user may receive relevant run data (e.g., confirmation of successful completion of the run, run specific metrics, etc.), as well as process specific information (e.g., amount of sample generated, presence or absence of specific target sequence, etc.). Data generated by the run may be subjected to subsequent bioinformatics analysis, which can be either local or cloud based. Depending on the process, a finished sample may be extracted from the cartridge or detection chip for subsequent use (e.g., genomic sequencing, qPCR quantification, cloning, etc.). The device may then be opened, and the cartridge or detection chip may then be removed.

In some embodiments, the module for biosensor detection comprises a pump. In some embodiments, the pump is peristaltic pump. Some such pumps comprise one or more of the inventive components for fluid handling described herein. For example, the pump may comprise an apparatus and/or a cartridge or detection chip. In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for certain of the advantages describe herein to be achieved (e.g., facile disengagement of the apparatus from the cartridge or detection chip, well-metered stroke volumes). In certain embodiments, the cartridge or detection chip of the pump comprises channels (e.g., microfluidic channels). In some embodiments, at least a portion of the channels of the cartridge or detection chip have certain cross-sectional shapes and/or surface layers that may contribute to any of a number of advantages described herein.

One non-limiting aspect of some cartridge or detection chips that may, in some cases, provide certain benefits is the inclusion of channels having certain cross-sectional shapes in the cartridge or detection chips. For example, in some embodiments, the cartridge or detection chip comprises v-shaped channels. One potentially convenient but non-limiting way to form such v-shaped channels is by molding or machining v-shaped grooves into the cartridge or detection chip. The recognized advantages of including a v-shaped channel (also referred to herein as a v-groove or a channel having a substantially triangularly-shaped cross-section) in certain embodiments in which a roller of the apparatus engages with the cartridge or detection chip to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, certain conventional cross sectional shapes of the channels, such as semi-circular, may require that the roller have a certain dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

In certain aspects, the cartridge or detection chips comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In certain embodiments, this pumping by interfacing a cartridge or detection chip (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In certain embodiments, a crank-and-rocker mechanism is incorporated into the apparatus to carry out this motion of the roller.

### Fluorescence Detection

Excitation light is provided to the sequencing device or module from one or more light sources external to the sequencing device or module. Optical components of the sequencing device or module may receive the excitation light from the light source and direct the light for example through waveguides towards the array of sample wells of the sequencing device or module and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a nucleic acid or protein (or pluralities thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing device or module may include an optical system for receiving excitation light and directing the excitation light among the sample well array. The optical system may include one or more grating couplers configured to couple excitation light to the sequencing device or module and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device or module by improving the uniformity of excitation light received by sample wells of the sequencing device or module. Examples of suitable components, e.g., for coupling excitation light to a sample well and/or directing emission light to a photodetector, to include in a sequencing device or module are described in U.S. patent application Ser. No. 14/821,688, filed Aug. 7, 2015, titled “INTEGRATED DEVICE FOR PROBING, DETECTING AND ANALYZING MOLECULES;” U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES;” and International Patent Application No.: PCT/US2020/039868, filed Jun. 26, 2020, titled “OPTICAL AND ELECTRICAL SECONDARY PATH REJECTION;” each of which are incorporated herein by reference in their entirety. Examples of suitable grating couplers and waveguides that may be implemented in the sequencing device or module are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device or module, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing device or module may be used to position and align an excitation source to the sequencing device or module. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing device or module may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device or module, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device or module, which may be connected to an instrument interfaced with the sequencing device or module. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device or module. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device or module, such as proper alignment and/or information obtained by readout signals from the photodetectors on the sequencing device or module. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device or module, and/or data generated from the readout signals of the photodetector.

In some embodiments, the instrument may include a processing device configured to analyze data received from one or more photodetectors of the sequencing device or module and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device or module.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target nucleic acid or a target protein) or a plurality of molecules present in a sample (e.g., a plurality of nucleic acids or a plurality of proteins), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing nucleic acids or proteins based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

### Sequencing Device or Module

Sequencing of nucleic acids or proteins in accordance with the instant disclosure, in some aspects, may be performed using a system that permits single molecule analysis. The system may include a sequencing device or module and an instrument configured to interface with the sequencing device or module. The sequencing device or module may include an array of pixels, where individual pixels include a sample well and at least one photodetector. The sample wells of the sequencing device or module may be formed on or through a surface of the sequencing device or module and be configured to receive a sample placed on the surface of the sequencing device or module.

In some embodiments, the sample wells are a component of a cartridge or detection chip (e.g., a disposable or single-use cartridge or detection chip) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g., a target nucleic acid or a target protein). In some embodiments, the number of molecules within a sample well may be distributed among the sample wells of the sequencing device or module such that some sample wells contain one molecule (e.g., a target nucleic acid or a target protein) while others contain zero, two, or a plurality of molecules.

### Molecular Barcodes

In some embodiments, methods provided herein comprise contacting a molecular barcode with a barcode recognition molecule that binds one or more sites on the molecular barcode. In some embodiments, a barcode recognition molecule binds one or more sites on a plurality of molecular barcodes. Accordingly, in some embodiments, a barcode recognition molecule can be used to decipher barcode content from a plurality of different single molecules in a mixture (e.g., different analytes comprising the same or different molecular barcodes). As an illustrative and non-limiting example, a multiplexed mixture can include a plurality of analytes attached to molecular barcodes. Some of these molecular barcodes can include a sample index that is indicative of sample origin for the analyte attached thereto, and a barcode recognition molecule that binds the sample index can be used to determine which analytes originated from the corresponding sample.

In some embodiments, a single-molecule construct includes a molecular barcode (e.g., kinetic barcode). In some embodiments, a molecular barcode of the disclosure is a nucleic acid barcode (e.g., a single-stranded nucleic acid). In some embodiments, a nucleic acid barcode comprises DNA, RNA, PNA, and/or LNA. In some embodiments, a molecular barcode is a polypeptide barcode.

In some embodiments, a molecular barcode comprises a series of index sequences. For example, in some embodiments, a molecular barcode is a nucleic acid barcode comprising a series of index sequences. In some embodiments, each index sequence is different from any other index sequence of the series. In some embodiments, at least two index sequences of the series are the same. In some embodiments, the series of index sequences corresponds to a series of barcode recognition molecule binding sites. In some embodiments, a barcode recognition molecule binds to a site on the molecular barcode comprising two index sequences of the series. In some embodiments, each index sequence provides different information with respect to barcode content.

Further, in some embodiments, a molecular barcode is attached to an analyte (e.g., a payload molecule, a detector molecule). In some embodiments, an analyte is derived from a biological or synthetic source. In some embodiments, an analyte is derived from a serum sample, a blood sample, a tissue sample, or a single cell. In some embodiments, an analyte is a biomolecule. In some embodiments, an analyte is a nucleic acid or a polypeptide. In some embodiments, an analyte is a nucleic acid aptamer, a protein, or a protein fragment. In some embodiments, an analyte is a small molecule, a metabolite, or an antibody. In some embodiments, a molecular barcode is attached to an analyte via a linker. In some embodiments, the linker comprises a cleavage site (e.g., a photocleavable site). Accordingly, in some embodiments, a single-molecule construct comprising a cleavage sequence would allow for the removal of the analyte to simplify loading and/or analysis on a substrate surface (e.g., a chip).

Also, in some embodiments, a molecular barcode comprises an attachment molecule. In some embodiments, an attachment molecule is any moiety or linkage group suitable for surface immobilization of the molecular barcode. In some embodiments, the attachment molecule comprises a covalent or non-covalent linkage group. In some embodiments, the attachment molecule comprises a biotin moiety. In some embodiments, the attachment molecule comprises a bis-biotin moiety. Linkage groups and other compositions and methods useful for surface immobilization are described in further detail elsewhere herein and are known in the art.

In some embodiments, a cleavage site is an optional component which may not be incorporated into a single-molecule construct depending on a desired implementation. In some embodiments, an attachment molecule can be adjacent to an analyte, such that a molecular barcode may be attached to a surface through the analyte. Examples of other configurations of single-molecule constructs and linkage strategies are provided elsewhere herein.

In some aspects, methods of the disclosure relate to a barcode deconvolution approach that involves deciphering molecular identity, sample origin, and/or location of a single molecule on an array. In some embodiments, methods provided herein are advantageously used to deconvolute molecular barcode information in a multiplexed sample. For example, methods of the disclosure can be applied to techniques for single-cell polypeptide sequencing. In some embodiments, the resulting single-molecule constructs can be analyzed by polypeptide sequencing (e.g., dynamic peptide sequencing) and barcode recognition in accordance with the disclosure.

In some embodiments, a label molecule described herein may be attached or linked to a barcode (e.g., a molecular barcode) as described herein. In some embodiments, a reference molecule described herein may be attached or linked to a barcode (e.g., a molecular barcode) as described herein. In some embodiments, an affinity agent as described herein may be attached or linked to a barcode (e.g., a molecular barcode) as described herein.

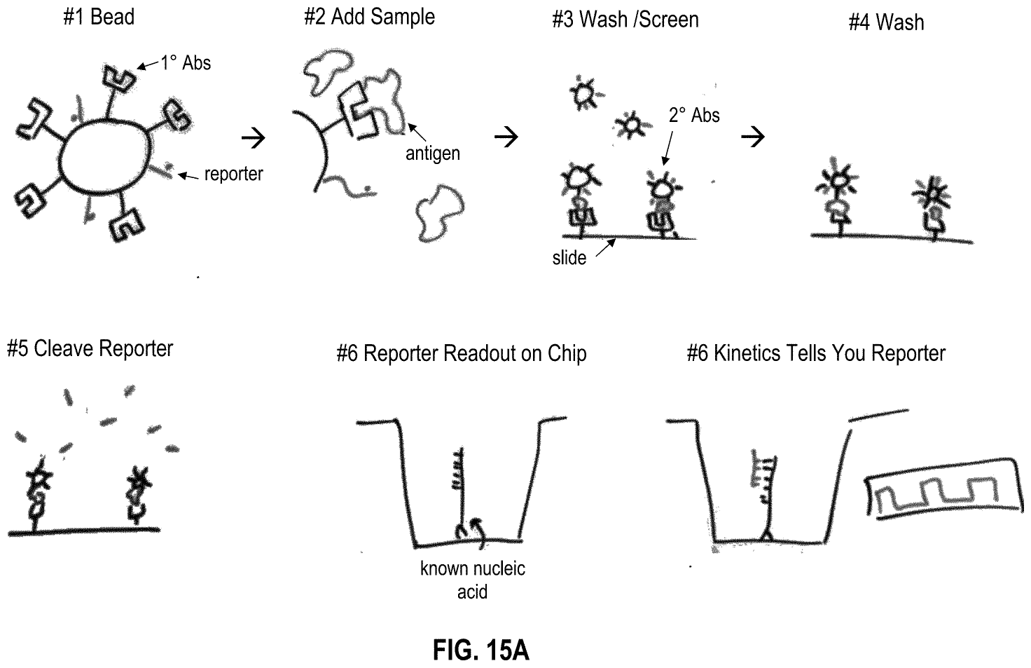

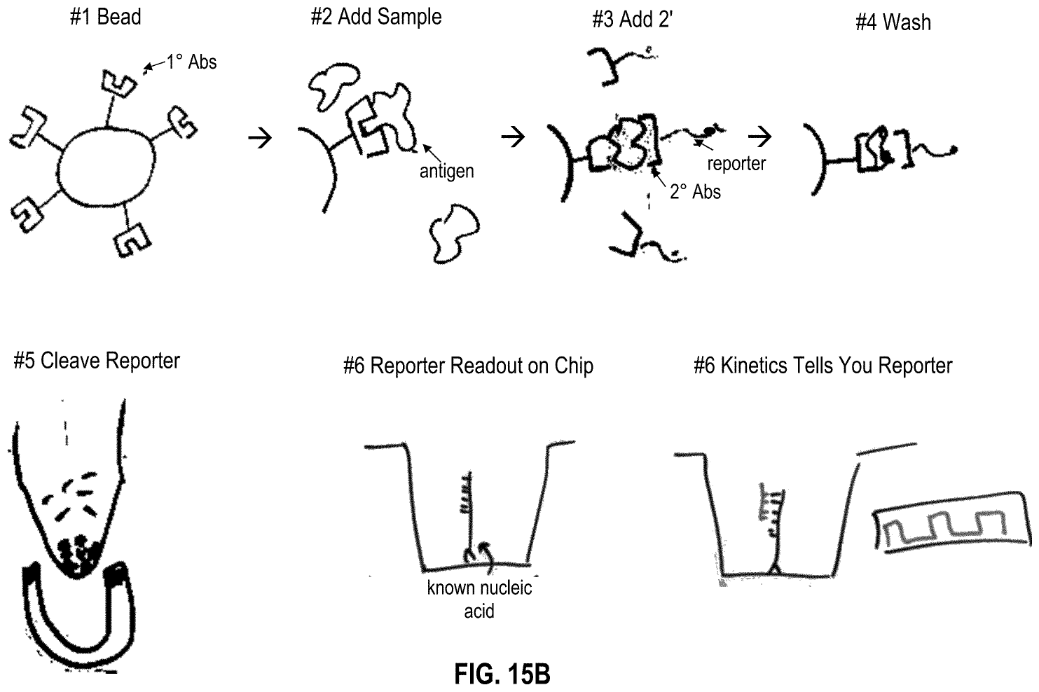

In some embodiments, a method of detecting a target molecule involves the use of workflow as described in FIG. 15A. For example, as shown in FIG. 15A, in a first step, a solid-phase bead (e.g., a magnetic bead) may be first loaded with primary antibodies (e.g., 1° antibodies) and a surface-associated label molecule (reporter). The label molecule may be a label nucleic acid. The label molecule may be attached or linked to a barcode (e.g., a molecular barcode) as described herein. In a second step, a sample comprising a target molecule (e.g., wherein the target molecule is an antigen) may be added to the beads comprising surface-immobilized antibodies. In some embodiments, the sample comprises an antigen that binds (e.g., specifically binds) to the surface-immobilized antibodies. In a third step, the beads may be added to a surface comprising surface-immobilized secondary antibodies (2° antibodies) to generate complexes comprising a target molecule bound to, and between, a surface-immobilized secondary antibody and a primary antibody attached to a bead, wherein the bead is linked to a surface-associated label molecule. In a fourth step, the complexes are washed using a wash solution or buffer. In a fifth step, the label molecules (reporters) are removed (or cleaved) and isolated from the beads. In a sixth step, the label molecules are added to a detection chip (e.g., comprising multiple sample wells), wherein the detection chip comprises a known molecule (e.g., a known nucleic acid or a “known readout oligo”) attached to the chip. The label molecule has a binding affinity to the known molecule. The binding associations and kinetics between the label molecule and the known molecule provide a determination of the identity of the label molecule. Such a determination of the identity of the label molecule allows for identification of the soluble antibodies (e.g., secondary antibodies (2° antibodies)) associated with that label molecule, which subsequently can allow for identification of the antigen capable of binding to the soluble antibodies.

In some embodiments, a method of detecting a target molecule involves the use of workflow as described in FIG. 15B. For example, as shown in FIG. 15B, in a first step, a magnetic solid-phase bead may be first loaded with surface-immobilized antibodies (e.g., primary antibodies (1° antibodies)). In a second step, a sample comprising a target molecule (e.g., wherein the target molecule is an antigen) may be added to the beads comprising surface-immobilized antibodies. In some embodiments, the sample comprises an antigen that binds (e.g., specifically binds) to the surface-immobilized antibodies. In a third step, soluble antibodies (e.g., secondary antibodies (2° antibodies)) linked to a label molecule (reporter) may be added to the beads to generate complexes comprising a target molecule bound to, and between, a surface-immobilized antibody and a soluble antibody linked to a label molecule, wherein the label molecule is optionally a nucleic acid. In a fourth step, the beads are washed using a wash solution or buffer. In a fifth step, the beads are isolated using a magnet and the label molecules (reporters) are removed (or cleaved) from the soluble antibodies. The label molecules are subsequently isolated. In a sixth step, the label molecules are added to a detection chip (e.g., comprising multiple sample wells), wherein the detection chip comprises a known molecule (e.g., a known nucleic acid or a “known readout oligo”) attached to the chip. The label molecule has a binding affinity to the known molecule. The binding associations and kinetics between the label molecule and the known molecule provide a determination of the identity of the label molecule. Such a determination of the identity of the label molecule allows for identification of the soluble antibodies (e.g., secondary antibodies (2° antibodies)) associated with that label molecule, which subsequently can allow for identification of the antigen capable of binding to the soluble antibodies. In some embodiments, the label molecule is attached or linked to a barcode (e.g., a molecular barcode) as described herein.

Aspects of the disclosure relate to identifying content of a molecular barcode. As used herein, “identifying,” “recognizing,” “recognition,” and like terms, in reference to a molecular barcode includes determination of partial identity (e.g., partial sequence information) as well as full identity (e.g., full sequence information) of the molecular barcode. In some embodiments, the terminology includes determining or inferring a nucleotide sequence of at least a portion of a molecular barcode (e.g., based on complementarity with an oligonucleotide probe). In yet other embodiments, the terminology includes determining or inferring a certain characteristics of a molecular barcode, such as the presence or absence of a particular index sequence at one or more sites on a molecular barcode. Accordingly, in some embodiments, the terms “barcode content,” “barcode identity,” and like terms as used herein may refer to qualitative information pertaining to a molecular barcode and are not restricted to the specific sequence information (e.g., the nucleotide sequence of an index) that biochemically characterizes a molecular barcode.

In some embodiments, barcode recognition is performed by observing different association events between a barcode recognition molecule and a molecular barcode, where each association event produces a change in magnitude of a signal that persists for a duration of time. In some embodiments, these changes in magnitude are detected as a series of signal pulses, or a series of pulses in a signal trace output. As described herein, signal pulse information may be used to identify barcode content based on a barcode-specific pattern in a series of signal pulses. In some embodiments, a barcode-specific pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a barcode-specific pattern. In some embodiments, the mean pulse duration of a barcode-specific pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different barcode-specific patterns corresponding to different barcode content may be distinguished from one another based on a statistically significant difference in the summary statistic. For example, in some embodiments, one barcode-specific pattern may be distinguishable from another barcode-specific pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one barcode-specific pattern is different from the mean pulse duration of another barcode-specific pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different barcode-specific patterns may require a greater number of pulse durations within each barcode-specific pattern to distinguish one from another with statistical confidence.

In some embodiments, a barcode-specific pattern generally refers to a plurality of association (e.g., binding) events between a barcode recognition molecule and a molecular barcode. In some embodiments, a barcode-specific pattern comprises at least 10 association events (e.g., at least 25, at least 50, at least 75, at least 100, at least 250, at least 500, at least 1,000, or more, association events). In some embodiments, a barcode-specific pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a barcode-specific pattern refers to a plurality of signal pulses which may be characterized by a summary statistic as described herein. In some embodiments, a barcode-specific pattern comprises at least 10 signal pulses (e.g., at least 25, at least 50, at least 75, at least 100, at least 250, at least 500, at least 1,000, or more, signal pulses). In some embodiments, a barcode-specific pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a barcode-specific pattern refers to a plurality of association (e.g., binding) events between a barcode recognition molecule and a molecular barcode occurring over a time interval. In some embodiments, barcode recognition may be carried out by iterative wash cycles in which molecular barcodes are exposed to different sets of barcode recognition molecules over different time durations. In some embodiments, the time interval of a barcode-specific pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, experimental conditions can be configured to achieve a time interval that allows for sufficient association events which provide a desired confidence level with a barcode-specific pattern (e.g., before a given set of barcode recognition molecules is removed during wash cycles). This can be achieved, for example, by configuring the reaction conditions based on various properties, including: reagent concentration, molar ratio of one reagent to another (e.g., ratio of barcode recognition molecule to molecular barcode, ratio of one barcode recognition molecule to another), number of different reagent types (e.g., the number of different types of barcode recognition molecules), binding properties (e.g., kinetic and/or thermodynamic binding parameters for barcode recognition molecule binding), reagent modification (e.g., polyol and other protein modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkage groups), surface modification (e.g., modification of sample well surface, including molecular barcode immobilization), sample preparation (e.g., analyte size, molecular barcode modification for immobilization), and other aspects described herein.

### Barcode Recognition Molecules

In some aspects, the disclosure provides barcode recognition molecules and methods of using the same. In some embodiments, a barcode recognition molecule can be selected or engineered based on desired binding kinetics with respect to a barcode site. For example, in some aspects, methods described herein can be performed in a multiplexed format in which a plurality of sites must be distinguished from one another based on binding interactions at each site. As such, the binding interactions at one site should be sufficiently different from binding interactions at another site, such that the different sites can be distinguished with higher confidence based on signal pulse information.

Without wishing to be bound by theory, a barcode recognition molecule binds to a barcode site according to a binding affinity (K_{D}) defined by an association rate, or an “on” rate, of binding (kₒₙ) and a dissociation rate, or an “off” rate, of binding (k_{off}). The rate constants k_{off} and kₒₙ are the critical determinants of pulse duration (e.g., the time corresponding to a detectable association event) and interpulse duration (e.g., the time between detectable association events), respectively. In some embodiments, these kinetic rate constants can be engineered to achieve pulse durations and pulse rates (e.g., the frequency of signal pulses) that give the best accuracy.

In some embodiments, a barcode recognition molecule may be engineered by one skilled in the art using conventionally known techniques. In some embodiments, desirable properties may include an ability to bind with low to moderate affinity (e.g., with a K_{D} of about 50 nM or higher, for example, between about 50 nM and about 50 μM, between about 100 nM and about 10 μM, between about 500 nM and about 50 μM) to one or more sites on a molecular barcode. For example, in some aspects, the disclosure provides methods of barcode recognition by detecting reversible binding interactions, and barcode recognition molecules that reversibly bind molecular barcodes with low to moderate affinity advantageously provide more informative binding data and with higher certainty than high affinity binding interactions.

In some embodiments, a barcode recognition molecule binds one or more sites on a molecular barcode with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other off-target (e.g., non-complementary) sites. In some embodiments, a barcode recognition molecule binds one or more sites on a molecular barcode with a K_{D} of less than about 100 nM, less than about 50 nM, less than about 25 nM, less than about 10 nM, or less than about 1 nM. In some embodiments, a barcode recognition molecule binds one or more sites on a molecular barcode with a K_{D} of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, a barcode recognition molecule binds one or more sites on a molecular barcode with a K_{D} of about 50 nM.

In some embodiments, a barcode recognition molecule binds one or more sites on a molecular barcode with a dissociation rate (k_{off}) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s⁻¹ and about 2 s⁻¹.

In some embodiments, the value for K_{D} or k_{off} can be a known literature value, or the value can be determined empirically. For example, the value for K_{D} or k_{off} can be measured in a single-molecule assay or an ensemble assay. In some embodiments, the value for k_{off} can be determined empirically based on signal pulse information obtained in a single-molecule assay as described elsewhere herein. For example, the value for k_{off} can be approximated by the reciprocal of the mean pulse duration. In some embodiments, a barcode recognition molecule binds two or more chemically different barcode sites with a different K_{D} or k_{off} for each of the two or more sites. In some embodiments, a first K_{D} or k_{off} for a first site differs from a second K_{D} or k_{off} for a second site by at least 10% (e.g., at least 25%, at least 50%, at least 100%, or more). In some embodiments, the first and second values for K_{D} or k_{off} differ by about 10-25%, 25-50%, 50-75%, 75-100%, or more than 100%, for example by about 2-fold, 3-fold, 4-fold, 5-fold, or more.

As described herein, a barcode recognition molecule may be any biomolecule capable of binding one or more sites on a molecular barcode over other barcode sites. Recognition molecules include, for example, oligonucleotides, nucleic acids, and proteins, any of which may be synthetic or recombinant.

In some embodiments, a barcode recognition molecule is an oligonucleotide (e.g., an oligonucleotide probe). In some embodiments, methods provided herein can be performed by contacting a nucleic acid barcode with an oligonucleotide probe that binds one or more sites on the nucleic acid barcode. In some embodiments, the binding between the oligonucleotide probe and the nucleic acid barcode occurs via hybridization or annealing. Beyond certain experimental conditions (e.g., concentration, temperature), binding properties are in large part driven by length and content of the oligonucleotide probe and its degree of complementarity with the site on the nucleic acid barcode to which it binds (e.g., hybridizes, or anneals). Accordingly, in some embodiments, oligonucleotide probes provide a variety of tunable features for modulating signal pulse characteristics, including, without limitation, length, nucleotide content (e.g., G/C content, nucleotide analogs with different binding characteristics, such as LNA or PNA analogs), degree of complementarity, and experimental factors, such as concentration, temperature, buffer conditions (e.g., pH, salt, magnesium), and DNA denaturing or stabilizing solvents.

In some embodiments, an oligonucleotide probe is at least four nucleotides in length. In some embodiments, an oligonucleotide probe is at least 5, at least 6, at least 7, at least 8, at least 9, at least 10, at least 12, at least 15, at least 20, at least 25, or at least 30 nucleotides in length. In some embodiments, an oligonucleotide probe is fewer than 30 nucleotides in length (e.g., fewer than 25, fewer than 20, fewer than 15, fewer than 12, fewer than 10 nucleotides in length). In some embodiments, an oligonucleotide probe is between about 3 and about 30 nucleotides in length (e.g., between about 3 and about 10, between about 3 and about 8, between about 5 and about 25, between about 5 and about 15, or between about 5 and 10 nucleotides in length). In some embodiments, an oligonucleotide probe is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an oligonucleotide probe can bind to, and provide barcode content information for, one or more barcode sites that are not fully complementary with the oligonucleotide probe. For example, in some embodiments, an oligonucleotide probe binds to one or more barcode sites having a sequence that is less than 100% complementary with the oligonucleotide (e.g., less than 99%, less than 98%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less).

In addition to oligonucleotides, nucleic acid aptamers can be used as barcode recognition molecules in accordance with the disclosure. Nucleic acid aptamers are nucleic acid molecules that have been engineered to bind targets with a desired affinity and selectivity. Accordingly, nucleic acid aptamers may be engineered to bind to a desired barcode site using selection and/or enrichment techniques known in the art. In some embodiments, a barcode recognition molecule comprises a nucleic acid aptamer, such as a DNA aptamer or an RNA aptamer.

In some embodiments, a barcode recognition molecule is a protein or polypeptide. In some embodiments, a recognition molecule is an antibody or an antigen-binding portion of an antibody, an SH2 domain-containing protein or fragment thereof, or an inactivated enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING.”

In some embodiments, a barcode recognition molecule is an amino acid recognition molecule. For example, in some embodiments, a molecular barcode comprises a polypeptide barcode, and an amino acid recognition molecule can be used to decipher barcode content from the polypeptide. In some embodiments, an amino acid recognition molecule binds one or more types of terminal amino acids with different kinetic binding properties. In some embodiments, an amino acid recognition molecule binds different segments of a polypeptide with different kinetic binding properties. For example, in some embodiments, an amino acid recognition molecule binds to polypeptide segments comprising the same type of amino acid at the N-terminus or C-terminus but differing in amino acid content at the penultimate (e.g., n+1) and/or subsequent positions (e.g., different amino acid types at one or more of the second, third, fourth, fifth, or higher, position) relative to the terminal amino acid. These concepts (e.g., differential binding kinetics based on differences in amino acid content only at the penultimate position or higher) and additional examples of amino acid recognition molecules are described more fully in

PCT International Publication No. WO2020102741A1, filed Nov. 15, 2019, titled “METHODS AND COMPOSITIONS FOR PROTEIN SEQUENCING,” which is incorporated by reference in its entirety.

In some embodiments, methods provided herein comprise contacting a molecular barcode with one or more barcode recognition molecules. For the purposes of this discussion, one or more barcode recognition molecules in the context of a method described herein may be alternatively referred to as a set of barcode recognition molecules. In some embodiments, a set of barcode recognition molecules comprises at least two and up to twenty (e.g., between 2 and 15, between 2 and 10, between 5 and 10, between 10 and 20) barcode recognition molecules. In some embodiments, a set of barcode recognition molecules comprises more than twenty (e.g., 20 to 25, 20 to 30) barcode recognition molecules. It should be appreciated, however, that any number of barcode recognition molecules may be used in accordance with a method of the disclosure to accommodate a desired use.

In accordance with the disclosure, in some embodiments, molecular barcode content can be identified by detecting luminescence from a label attached to a barcode recognition molecule. In some embodiments, a labeled barcode recognition molecule comprises a barcode recognition molecule that binds at least one molecular barcode and a luminescent label having a luminescence that is associated with the barcode recognition molecule. In this way, the luminescence (e.g., luminescence lifetime, luminescence intensity, and other luminescence properties described elsewhere herein, including luminescence-based kinetic binding data) may be associated with the binding of the barcode recognition molecule to identify the at least one molecular barcode. In some embodiments, a plurality of types of labeled barcode recognition molecules may be used in a method according to the disclosure, wherein each type comprises a luminescent label having a luminescence that is uniquely identifiable from among the plurality. Suitable luminescent labels may include luminescent molecules, such as fluorophore dyes, and are described elsewhere herein.

In some embodiments, a barcode recognition molecule comprises a label having binding-induced luminescence. For example, in some embodiments, a labeled aptamer can comprise a donor label and an acceptor label. As a free and unbound molecule, the labeled aptamer adopts a conformation in which the donor and acceptor labels are separated by a distance that limits detectable FRET between the labels (e.g., about 10 nm or more). Upon binding to a barcode site, the labeled aptamer adopts a conformation in which the donor and acceptor labels are within a distance that promotes detectable FRET between the labels (e.g., about 10 nm or less). In yet other embodiments, a labeled aptamer can comprise a quenching moiety and function analogously to a molecular beacon, wherein luminescence is internally quenched as a free molecule and restored upon binding to a barcode site (see, e.g., Hamaguchi, et al. (2001) Analytical Biochemistry 294, 126-131). Similar and alternative labeling strategies would be apparent to those skilled in the art, such as the use of FRET between a labeled aptamer and a labeled molecular barcode. Without wishing to be bound by theory, it is thought that these and other types of mechanisms for binding-induced luminescence may advantageously reduce or eliminate background luminescence to increase overall sensitivity and accuracy of the methods described herein.

In some embodiments, molecular barcode content can be identified by detecting one or more electrical characteristics of a labeled barcode recognition molecule. In some embodiments, a labeled barcode recognition molecule comprises a barcode recognition molecule that binds at least one molecular barcode and a conductivity label that is associated with the barcode recognition molecule. In this way, the one or more electrical characteristics (e.g., charge, current oscillation color, and other electrical characteristics, including conductivity-based kinetic binding data) may be associated with the binding of the barcode recognition molecule to identify the at least one molecular barcode. In some embodiments, a plurality of types of labeled barcode recognition molecules may be used in a method according to the disclosure, wherein each type comprises a conductivity label that produces a change in an electrical signal (e.g., a change in conductance, such as a change in amplitude of conductivity and conductivity transitions of a barcode-specific pattern) that is uniquely identifiable from among the plurality. In some embodiments, the plurality of types of labeled barcode recognition molecules each comprises a conductivity label having a different number of charged groups (e.g., a different number of negatively and/or positively charged groups). Accordingly, in some embodiments, a conductivity label is a charge label. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

### EXAMPLES

Embodiments of the disclosure are further described with reference to the following examples, which are intended to be illustrative and not restrictive in nature.

### Example 1. Preparation of of Streptavidin-immobilized PLL Surfaces and Detection of Binding Events of Dye-labeled dsDNA Molecules at Low Concentration

The detection chips shown in FIG. 8 (e.g., comprising positively-charged molecules functionalized to the interior base of each sample well) were generated as follows:
1. A complementary metal-oxide-semiconductor (CMOS) chip containing nanoapertures functionalized at the bottom surface with Biotin-PEG-silane was wetted with 70% isopropanol (3 times), 0.1% Tween-20 (3 times), and 3 times with 1× binding buffer (50 mM MOPS, 75 mM KOAc, 10 mM DTT, 0.03% Tween-20, pH 7.5).
2. The chip was incubated with 20 nM streptavidin in 1× binding buffer at room temperature for 30 min.
3. The chip was washed 5 times with buffer SC-6 buffer (NaCl 200-350 mM, KCl 10-30 mM, Na2HPO4 3 mM, KHPO4 1 mM, 4-Nitrobenzoic acid 5 mM, D-glucose 50 mM, 0.1% Tween-20, pH=7.5).
4. The chip was functionalized by adding 100 nM of biotin-poly-Lysine (PLL) in SC-6, incubating at room temperature for 30 min, and washing 5-times with SC-6. Detection chips produced by this method provided an average 2-3 biotins per 1000 nm² and subsequent polylysine attachment provided ˜40-300 positive charges per 1000 nm² depending on the length of polylysine used.
5. Reaction buffer (SC-6 buffer containing an oxygen scavenging system) was added to the chip and a detection run was initiated.
6. After 10 minutes of detection, a 25 bp Atto-Rho6G labeled dsDNA molecule was added to the reaction buffer at a final concentration of 25 pM and detection was continued for an additional 50 minutes.
7. Traces collected from the detection run displayed a clear pattern of pulsing behavior that initiated upon addition of the dye-labeled dsDNA molecule, demonstrating that chips prepared in this manner successfully detect reversible binding events of single dye-labeled DNA molecules at very low concentration.

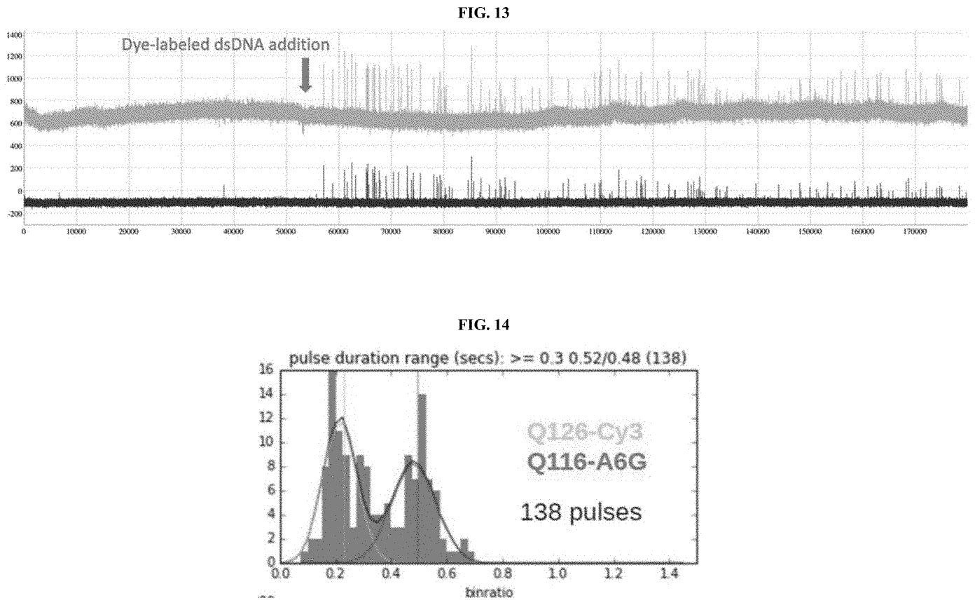

An example trace collected from a detection run is depicted in FIG. 13. This example trace (split between two time bins produced by the fluorescence lifetime sensitive operation of the CMOS chip) shows the initiation of pulsing upon addition of the labeled dsDNA due to reversible binding of the dsDNA to the PLL functionalized surface at the bottom of the nanoaperture.

### Example 2. Preparation of Streptavidin-immobilized PLL Surfaces, Detection of Differentially Labeled dsDNA Molecules at Low Concentration, and Determination of Dye Ratios

Detection chips produced by the method in Example 1 were used to demonstrate detection of the ratio of dye-labeled dsDNA molecules at very low concentration as follows:
1. Two types of dsDNA molecules (20-40 bp) containing distinguishable fluorophores were prepared separately (one containing Cy3 and the other containing Atto-Rho6G which are distinguishable by fluorescence lifetime on the Quantum-Si CMOS chip). The two types of labeled dsDNA molecules were mixed in a 1:1 ratio and diluted in reaction buffer (SC-6 buffer containing an oxygen scavenging system) to a final concentration of 25 pM each.
2. The sample was added to a CMOS chip prepared as in Example 1 and a detection run was performed for 1 hour.
3. The data collected from the detection run consisted of traces containing signal pulses corresponding to reversible binding events of the labeled dsDNA molecules to the positively-charged PLL surface. Signal pulses were identified and each pulse was assigned to Cy3 or Atto-Rho6G based on the fluorescence lifetime information detected by the CMOS chip during the pulse.

In agreement with the 1:1 mixing ratio of the two types of labeled dsDNA molecules, Cy3 pulses and Atto-Rho6G pulses were determined to be present in approximately equal amounts (52% to 48%) as shown in FIG. 14. The ‘binratio’ (a measure of the relative signal collected in the lifetime-sensitive time bins of the CMOS sensor that corresponds to fluorescence lifetimes of the dyes) was determined for each pulse and a gaussian mixture model applied to the binratio distribution was used to classify each pulse as a detection event for Cy3 or Atto-Rho6G.

### Example 3. Detection of Reversible Binding Events of Dye-labeled dsDNA Molecules on Chips Comprising Surfaces Directly Coupled with a Positively-charged Amine-terminated Silane

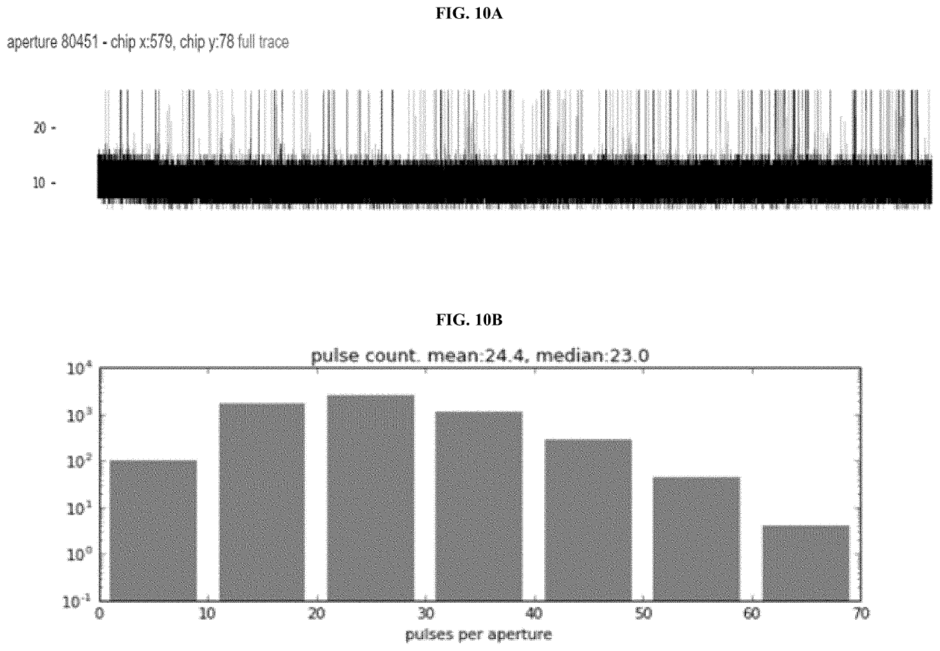

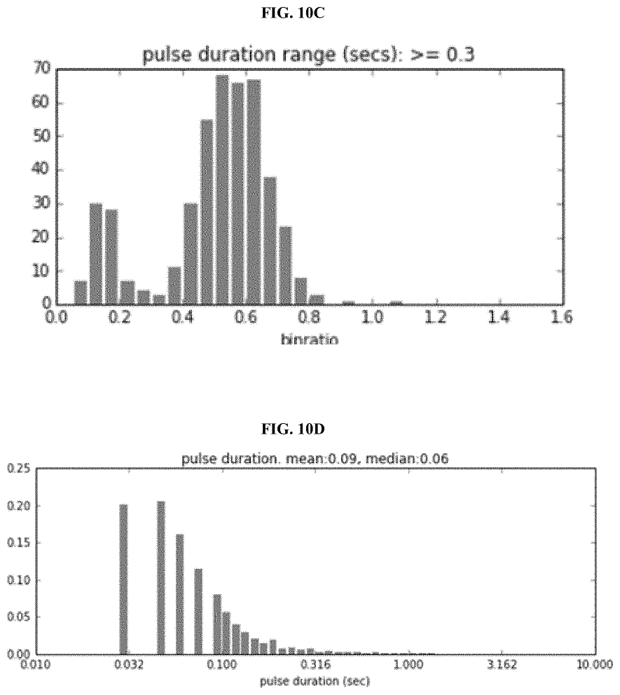

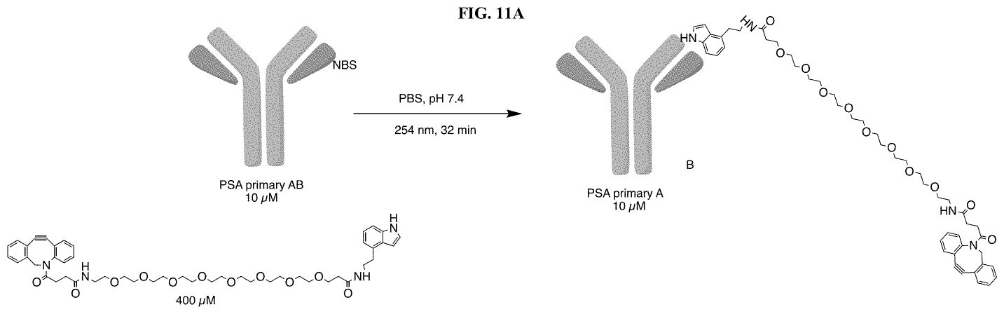

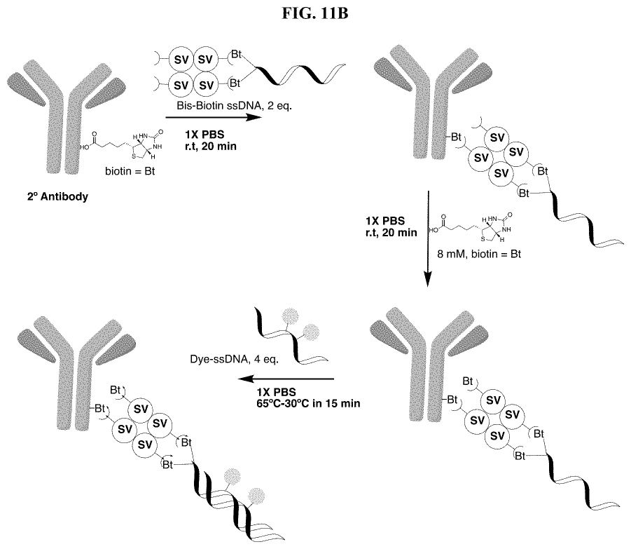

Detection chips comprising positively-charged amine-terminated silane were prepared as follows:
1. A CMOS chip was prepared via silane surface passivation chemistry with the amine-terminated silane molecule depicted in FIG. 9. The procedure resulted in direct coupling of the positively-charged amine terminated molecules to the glass surfaces located at the bottom of the nanoapertures (also depicted in FIG. 9.).
2. The chip was wetted with 70% isopropanol (3 times), 0.1% Tween-20 (3 times) and washed 5 times with buffer SC-6.
3. Two types of dsDNA molecules (20-40 bp) containing distinguishable fluorophores were prepared separately (one containing Cy3 and the other containing Atto-Rho6G which are distinguishable by fluorescence lifetime on the Quantum-Si CMOS chip). The two types of labeled dsDNA molecules were mixed in a 1:1 ratio and diluted in reaction buffer (SC-6 buffer containing an oxygen scavenging system) to a final concentration of 25 pM each.
4. The sample was added to a CMOS chip and a detection run was performed for 1 hour.
5. Successful dynamic pulsing behavior was observed. See FIGS. 10A-10D for representative data collected using these detection chips.

Detection chips produced by this above method provided limited charge density by available aperture opening area with an estimated charge density at 80-120 counts per 1000 nm².

### Further Aspects of the Disclosure

Aspects of the exemplary embodiments and examples described above may be combined in various combinations and subcombinations to yield further embodiments of the disclosure. To the extent that aspects of the exemplary embodiments and examples described above are not mutually exclusive, it is intended that all such combinations and subcombinations are within the scope of the present disclosure. It will be apparent to those of skill in the art that embodiments of the present disclosure include a number of aspects. Accordingly, the scope of the claims should not be limited by the preferred embodiments set forth in the description and examples, but should be given the broadest interpretation consistent with the description as a whole.

What is claimed is:

1. A method of determining the concentration of target molecules in a sample, the method comprising:
(i) contacting the sample comprising the target molecules with a plurality of first affinity agents having a binding affinity for the target molecules to produce a plurality of first complexes comprising a target molecule bound to a first affinity agent, wherein at least a portion of the plurality of first affinity agents are immobilized to a surface;
(ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules;
(iii) optionally removing unbound second affinity agents and/or isolating the plurality of second complexes;
(iv) optionally isolating at least a segment of each of the label molecules from the bound second affinity agents of the plurality of second complexes;
(v) combining the segments of the label molecules with a known concentration of reference molecules;
(vi) determining the ratio of detection events of label molecules relative to detection events of reference molecules; and
(vii) determining the concentration of target molecules in the sample based at least in part on the ratio of detection events of label molecules relative to detection events of reference molecules.

2. A method of determining the concentration of target molecules in a sample, the method comprising:
(i)(a) contacting the sample comprising the target molecules with a plurality of first affinity agents having a binding affinity for the target molecules to produce a plurality of first complexes comprising a target molecule bound to a first affinity agent,
(i)(b) immobilizing at least a portion of the plurality of first affinity agents to a surface;
(ii) contacting the plurality of first complexes with a plurality of second affinity agents having a binding affinity for the first complexes to produce a plurality of second complexes comprising a second affinity agent bound to a first complex, wherein at least a portion of the second affinity agents are linked to label molecules;
(iii) optionally removing unbound second affinity agents and/or isolating the plurality of second complexes;
(iv) optionally isolating at least a segment of each of the label molecules from the bound second affinity agents of the plurality of second complexes;
(v) combining the isolated segments of the label molecules with a known concentration of reference molecules;
(vi) determining the ratio of detection events of label molecules relative to detection events of reference molecules; and
(vii) determining the concentration of target molecules in the sample based at least in part on the ratio of detection events of label molecules relative to detection events of reference molecules.

2. The method of claim 1, wherein the target molecules are proteins, small molecules, or nucleic acids.

3. The method of claim 1, wherein the contacting of (i) and/or (ii) is performed at a temperature of 4-37° C., optionally 4-25° C.; and/or the contacting of (i) and/or (ii) is performed for 5 minutes to 4 hours.

4. The method of claim 1, wherein the first affinity agents are antibodies or aptamers, the target molecules are proteins, the antibodies or aptamers specifically bind to an epitope of the target molecules, and the second affinity agents are antibodies.

5. The method of claim 1, wherein the at least a portion of the plurality of first affinity agents are immobilized to the surface of a solid-phase bead, microfluidic channel, nanoaperture, resin, matrix, membrane, polymer, plastic, metallic, or glass.

6. The method of claim 1, wherein each of the label molecules is linked to at least one fluorophore.

7. The method of claim 1, wherein each of the label molecules is a label nucleic acid, wherein each of the second affinity agents is linked to a first strand of the label nucleic acid, and optionally wherein the first strand is 5-50 nucleobases in length.

8. The method of claim 7, wherein the label nucleic acid is a single-stranded nucleic acid and/or comprises a region that forms a hairpin loop and/or is linked to at least one fluorophore.

9. The method of claim 8, wherein the label nucleic acid is a double-stranded nucleic acid comprising the first strand and a second strand comprising a region of complementarity to the first strand.

10. The method of claim 1, wherein (i) and (ii) occur simultaneously or in series; and/or wherein (iii) comprises removing the unbound second affinity agents by washing the sample with a wash buffer.

11. The method of claim 1, wherein (iv) comprises isolating the at least a segment of each of the label molecules from the bound second affinity agents by washing the sample with an elution buffer and/or wherein (iv) comprises isolating the at least a segment of each of the label molecules from the bound second affinity agents by altering the temperature (e.g., increasing the temperature) of the sample.

12. The method of claim 6, wherein each of the reference molecules is linked to at least one fluorophore, wherein the fluorophore linked to a reference molecule is distinct from at least one fluorophore linked to a label molecule.

13. The method of claim 12, wherein the at least one fluorophore linked to a reference molecule and the at least one fluorophore linked to a label molecule can be excited by the same excitation wavelength.

14. The method of claim 7, wherein each of the reference molecules is a reference nucleic acid.

15. The method of claim 1, wherein each of the reference molecules are
(a) immobilized to the same surface as the at least a portion of the plurality of first affinity agents;
(b) immobilized to a surface that is different from the surface to which the at least a portion of the plurality of first affinity agents are immobilized; or
(c) linked to a first affinity agent.

16. The method of claim 1, wherein the isolated segments of the label molecules and the known concentration of reference molecules are combined in (v) in a detection chip, optionally wherein the detection chip comprises an ordered array of sample wells.

17. The method of claim 16, wherein the interior base of each sample well is functionalized with:
(a) a silane-containing compound;
(b) a biotin-streptavidin complex;
(c) positively-charged molecules, optionally polylysine molecules; or
(d) nucleic acids that are complementary to the label molecules and/or reference molecules, wherein the label molecules and reference molecules are nucleic acids.

18. The method of claim 1, wherein the ratio of label molecules relative to reference molecules is determined using fluorescence measurements and/or in part based on the dwell time of label and reference molecules in sample wells of a detection chip.

19. The method of claim 17, wherein the label and reference molecules are:
(I) delivered to and maintained in the sample wells by electrostatic interactions with positively-charged molecules at the interior base of each sample well;
(II) delivered to and maintained in the sample wells by interactions with nucleic acids at the interior base of each sample well that are complementary to the label nucleic acids and/or reference nucleic acids;
(III) delivered to and maintained in the sample wells by gravity or a magnetic field; or
(IV) delivered to and maintained in the sample wells using a crowding reagent, optionally wherein the crowding reagent is a sugar molecule, methylcellulose, polyethylene glycol, dextran, ficoll, bovine serum albumin, or trehalose.

20. The method of claim 1, wherein the labeled target molecules or label molecules further comprise a molecular barcode.

21. A detection chip comprising an array of sample wells, wherein a base of each sample well is functionalized with positively-charged molecules, optionally wherein the base is an interior base.

22. A method of determining the concentration of labeled target molecules in a sample, the method comprising:
combining the sample containing labeled target molecules with a known concentration of reference molecules;
determining the ratio of detection events of labeled target molecules relative to detection events of reference molecules; and
determining the concentration of labeled target molecules in the sample based at least in part on the ratio of detection events of labeled target molecules relative to detection events of reference molecules.

23. A method of determining the identity of a target molecule, the method comprising:
(A)
(i) contacting the target molecule with a first affinity agent having a binding affinity for the target molecule to produce a first complex comprising the target molecule bound to the first affinity agent, wherein the first affinity agent is immobilized to the surface of a solid-phase bead, and wherein a label molecule is attached to the surface of the solid-phase bead;
(ii) contacting the first complex with a surface-immobilized second affinity agent having a binding affinity for the target molecule to produce a second complex comprising a second affinity agent bound to the first complex;
(iii) optionally washing the second complex;
(iv) isolating the label molecule;
(v) contacting the isolated label molecule with a detection chip comprising a sample well, wherein a known molecule is attached to the sample well; and
(vi) determining the identity of the label molecule using fluorescence, luminescence, and/or kinetic measurements, thereby identifying the target molecule; or
(B)
(i) contacting the target molecule with a first affinity agent having a binding affinity for the target molecule to produce a first complex comprising the target molecule bound to the first affinity agent, wherein the first affinity agent is immobilized to the surface of a solid-phase bead;
(ii) contacting the first complex with a second affinity agent having a binding affinity for the target molecule to produce a second complex comprising a second affinity agent bound to the first complex, wherein the second affinity agent is attached to a label molecule;
(iii) optionally washing the second complex;
(iv) isolating the label molecule;
(v) contacting the isolated label molecule with a detection chip comprising a sample well, wherein a known molecule is attached to the sample well; and
(vi) determining the identity of the label molecule using fluorescence, luminescence, and/or kinetic measurements, thereby identifying the target molecule.
